(19) United States
(12) Patent Application Publication
(10) Pub. No.: US 2026/0159526 A1
(43) Pub. Date: Jun. 11, 2026

(54) CRYSTALLINE FORMS OF A MACROCYCLIC COMPOUND

(71) Applicant: Turning Point Therapeutics, Inc., Princeton, NJ (US)

(72) Inventors: Kevin MARTIN, Princeton, NJ (US); Yuelie LU, Princeton, NJ (US); Pengjian LIU, Shanghai (CN); Xiaoyang WANG, Shanghai (CN)

(21) Appl. No.: 19/181,635

(22) Filed: Apr. 17, 2025

(30) Foreign Application Priority Data
Apr. 19, 2024 (WO) PCT/CN2024/088941

(51) Int. Cl. C07D 498/18; A61K 31/529; C07C 59/255; C07C 309/35

(52) U.S. Cl. CPC C07D 498/18; A61K 31/529; C07C 59/255; C07C 309/35; C07B 2200/13

(57) ABSTRACT

The present disclosure provides crystalline forms of a compound of Formula (I) or a pharmaceutically acceptable salt or solvate thereof.

### CROSS REFERENCE TO RELATED APPLICATIONS

This application claims priority under 35 U.S.C. § 119 to International Patent Application No. PCT/CN2024/088941 filed Apr. 19, 2024, which is incorporated herein by reference in its entirety.

### BACKGROUND

Protein kinases regulate various functions in the cell including cell growth, proliferation and survival. Dysregulation of protein kinases is often the cause of many solid malignancies. The use of protein kinase inhibitors has led to substantial clinical benefit in patients harboring oncogenic aberrations. Protein kinase inhibitors have been approved for clinical treatment of cancers. RET is a receptor tyrosine kinase that is expressed with its highest levels in early embryogenesis (during which it has diverse roles in different tissues) and decreases to relatively low levels in normal adult tissues (Pachnis, V., et al. Development 1993, 119, 1005-1017). RET activation regulates the downstream signalling pathways (RAS/MAPK/ERK, PI3K/AKT, and JAK-STAT etc.), leading to cellular proliferation, migration, and differentiation (Mulligan, LM. Nat Rev Cancer. 2014, 14(3):173-86).

Elzovantinib (also known as TPX-0022 and (S,1³E,1⁴E)-1²-amino-2-ethyl-4⁵-fluoro-6-methyl-9-oxo-5-oxa-2,8-diaza-1(5,3)-pyrazolo[1,5-a]pyrimidina-4(1,2)-benzenacyclononaphane-4⁶-carbonitrile) has been reported as a potent inhibitor of SRC, MET, and CSF1R. See, Table 1 in U.S. Pat. Nos. 11,155,563, 11,286,264, reporting SRC IC₅₀=0.12 nM, MET IC₅₀=0.14 nM, and CSF1R IC₅₀=0.76 nM for Compound 5 in enzymatic kinase assays. Accordingly, the compound is believed to be useful in treating certain cancers, such as MET-driven advanced solid tumors.

Crystalline forms of a pharmaceutically acceptable salt or solvate of elzovantinib may present advantages in manufacturing, processing, or stability. The present disclosure addresses this and other needs.

### BRIEF SUMMARY

In some embodiments, a crystalline form of the present disclosure is a crystalline form of a pharmaceutically acceptable salt or solvate of a compound of Formula (I):

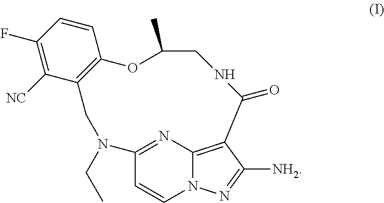 [SMILES: CCN1Cc2c(ccc(F)c2C#N)O[C@@H](C)CNC(=O)c2c(N)nn3ccc1nc23]

In some embodiments, a pharmaceutical composition comprises the crystalline form of the present disclosure, and a pharmaceutically acceptable carrier.

In some embodiments, a process for preparing a crystalline form of the present disclosure comprises: (a) combining a compound of Formula (I), 1, 5-naphthalenedisulfonic acid, and an alcohol to form a mixture; and (b) cooling the mixture; thereby preparing the crystalline form.

In some embodiments, a process for preparing a crystalline form of the present disclosure comprises combining a compound of Formula (I), L-tartaric acid, and an alcohol to form a mixture; thereby preparing the crystalline form.

In some embodiments, a process for preparing a crystalline form of the present disclosure comprises combining a 1, 5-naphthalenedisulfonic acid salt of a compound of Formula (I) and dichloromethane, thereby preparing the crystalline form.

In some embodiments, a method of the present disclosure comprises administering a crystalline form of a compound of Formula (I) or a pharmaceutical composition of the present disclosure.

### BRIEF DESCRIPTION OF THE DRAWINGS

FIG. 1 shows an X-ray powder diffraction (XRPD) pattern for a hemi 1, 5-naphthalenedisulfonic acid salt Form A of the compound of Formula (I).

FIG. 2 shows a differential scanning calorimetry (DSC) graph for a hemi 1, 5-naphthalenedisulfonic acid salt Form A of the compound of Formula (I).

FIG. 3 shows a thermogravimetric analysis (TGA) thermogram for a hemi 1, 5-naphthalenedisulfonic acid salt Form A of the compound of Formula (I).

FIG. 4 shows a dynamic vapor sorption (DVS) isotherm for a hemi 1, 5-naphthalenedisulfonic acid salt hydrate of the compound of Formula (I).

FIG. 5 shows an XRPD pattern for L-tartrate Form A of the compound of Formula (I).

FIG. 6 shows a DSC graph for L-tartrate Form A of the compound of Formula (I).

FIG. 7 shows a TGA thermogram for L-tartrate Form A of the compound of Formula (I).

FIG. 8 shows XRPD patterns for L-tartrate Form B wet (top graph), dried (middle graph), in comparison with L-tartrate Form A (bottom graph) of the compound of Formula (I).

FIG. 9 shows a DSC graph for L-tartrate Form B of the compound of Formula (I).

FIG. 10 shows a TGA thermogram for L-tartrate Form B of the compound of Formula (I).

FIG. 11 shows XRPD patterns for L-tartrate Form C wet (top graph), dried (middle graph), in comparison with L-tartrate Form A (bottom graph) of the compound of Formula (I).

FIG. 12 shows a DSC graph for L-tartrate Form C of the compound of Formula (I).

FIG. 13 shows a TGA thermogram for L-tartrate Form C of the compound of Formula (I).

FIG. 14 shows XRPD patterns for L-tartrate Form D wet (top graph), dried (middle graph), in comparison with L-tartrate Form A (bottom graph) of the compound of Formula (I).

FIG. 15 shows a DSC graph for L-tartrate Form D of the compound of Formula (I).

FIG. 16 shows a TGA thermogram for L-tartrate Form D of the compound of Formula (I).

FIG. 17 shows XRPD patterns for L-tartrate Form E wet (top graph), dried (middle graph), in comparison with L-tartrate Form A (bottom graph) of the compound of Formula (I).

FIG. 18 shows a DSC graph for L-tartrate Form E of the compound of Formula (I).

FIG. 19 shows a TGA thermogram for L-tartrate Form E of the compound of Formula (I).

FIG. 20 shows XRPD patterns for L-tartrate Form F wet (top graph), dried (middle graph), in comparison with L-tartrate Form A (bottom graph) of the compound of Formula (I).

FIG. 21 shows a DSC graph for L-tartrate Form F of the compound of Formula (I).

FIG. 22 shows a TGA thermogram for L-tartrate Form F of the compound of Formula (I).

FIG. 23 shows an XRPD pattern for a hydrochloride salt of the compound of Formula (I).

FIG. 24 shows an XRPD pattern for sulfate salt Form A of the compound of Formula (I).

FIG. 25 shows an XRPD pattern for sulfate salt Form B of the compound of Formula (I).

FIG. 26 shows an XRPD pattern for a methyl tert-butyl ether (MTBE) solvate of the compound of Formula (I).

FIG. 27 shows an XRPD pattern for a heptane solvate of the compound of Formula (I).

FIG. 28 shows an XRPD pattern for a cyclohexane solvate of the compound of Formula (I).

FIG. 29 shows an XRPD pattern for a methylcyclohexane solvate of the compound of Formula (I).

FIG. 30 shows an XRPD pattern for an ethyl acetate solvate of the compound of Formula (I).

FIG. 31 shows an XRPD pattern for an ethanol solvate of the compound of Formula (I).

FIG. 32 shows an XRPD pattern for an isopropanol solvate of the compound of Formula (I).

FIG. 33 shows an XRPD pattern for a dimethylsulfoxide (DMSO) solvate of the compound of Formula (I).

FIG. 34 shows an XRPD pattern for a toluene solvate of the compound of Formula (I).

FIG. 35 shows an XRPD pattern for a methyl acetate solvate of the compound of Formula (I).

FIG. 36 shows an XRPD pattern for a methyl isobutyl ketone solvate Form A of the compound of Formula (I).

FIG. 37 shows an XRPD pattern for a methyl isobutyl ketone solvate Form B of the compound of Formula (I).

FIG. 38 shows an XRPD pattern for a tetrahydrofuran (THF) solvate of the compound of Formula (I).

FIG. 39 shows an XRPD pattern for a dichloromethane (DCM) solvate Form A of the compound of Formula (I).

FIG. 40 shows an XRPD pattern for an acetonitrile solvate of the compound of Formula (I).

FIG. 41 shows an XRPD pattern for an acetone solvate of the compound of Formula (I).

FIG. 42 shows an XRPD pattern for a 2-methyltetrahydrofuran solvate of the compound of Formula (I).

FIG. 43 shows an XRPD pattern for a methyl ethyl ketone solvate of the compound of Formula (I).

FIG. 44 shows an XRPD pattern for a dichloromethane (DCM) solvate Form B of the compound of Formula (I).

FIG. 45 shows an XRPD pattern for a dimethylformamide (DMF) solvate of the compound of Formula (I).

FIG. 46 shows an XRPD pattern for an N-methylpyrrolidone (NMP) solvate of the compound of Formula (I).

FIG. 47 shows an ORTEP diagram for a dichloromethane solvate of a hemi 1, 5-naphthalenedisulfonic acid salt of the compound of Formula (I).

### DETAILED DESCRIPTION

In some embodiments, a crystalline form of the present disclosure is a crystalline form of a pharmaceutically acceptable salt or solvate of a compound of Formula (I):

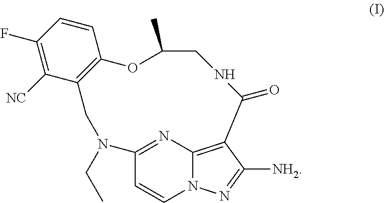 [SMILES: CCN1Cc2c(ccc(F)c2C#N)O[C@@H](C)CNC(=O)c2c(N)nn3ccc1nc23]

Such crystalline forms may provide ease of purification or other advantages such as ease of processing or stability as compared to the parent compound.

### I. DEFINITIONS

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof.

The definitions set forth herein take precedence over definitions set forth in any patent, patent application, and/or patent application publication incorporated herein by reference.

Reference to “about” a value or parameter herein includes (and describes) embodiments that are directed to that value or parameter per se. For example, description referring to “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

All measurements are subject to experimental error and are within the spirit of the invention.

As used herein, “hydrate” refers to a crystalline form of a molecule that further comprises water incorporated into the crystalline structure. The water molecules in the hydrate may be present in a regular arrangement and/or a non-ordered arrangement. The hydrate may comprise either a stoichiometric or nonstoichiometric amount of the water molecules, for example, monohydrate, hemihydrate, or a mixture thereof.

As used herein, “room temperature” is 25° C., unless otherwise indicated.

As used herein “solvate” refers to a crystalline form of a molecule, atom, and/or ions that further comprises molecules of a solvent or solvents incorporated into the crystalline lattice structure. The solvent molecules in the solvate may be present in a regular arrangement and/or a non-ordered arrangement. The solvate may comprise either a stoichiometric or nonstoichiometric amount of the solvent molecules.

As used herein, “substantially pure,” when used in reference to a crystalline form, means a compound having a purity greater than 90 weight %, including greater than 90, 91, 92, 93, 94, 95, 96, 97, 98, and 99 weight %, and also including equal to about 100 weight % of Compound (I), based on the weight of the compound. The remaining material comprises other form(s) of the compound, and/or reaction impurities and/or processing impurities arising from its preparation. For example, a crystalline form of Compound (I) may be deemed substantially pure in that it has a purity greater than 90 weight %, as measured by means that are at this time known and generally accepted in the art, where the remaining less than 10 weight % of material comprises amorphous and/or other form(s) of Compound (I) and/or reaction impurities and/or processing impurities.

As used herein, an XRPD pattern “comprising” a number of peaks selected from a specified group of peaks, is intended to include XRPD patterns having additional peaks that are not included in the specified group of peaks. For example, an XRPD pattern comprising four or more, preferably five or more, 20 values selected from: 6.8, 9.3, 10.2, 10.9, 12.8, 13.4, 14.4, 15.1, 21.6, and 25.3, is intended to include a XRPD pattern having: (a) four or more, preferably five or more, 20 values selected from: 6.8, 9.3, 10.2, 10.9, 12.8, 13.4, 14.4, 15.1, 21.6, and 25.3; and (b) zero or more peaks that are not one of peaks 6.8, 9.3, 10.2, 10.9, 12.8, 13.4, 14.4, 15.1, 21.6, and 25.3.

The presence of reaction impurities and/or processing impurities may be determined by analytical techniques known in the art, such as, for example, chromatography, nuclear magnetic resonance spectroscopy, mass spectrometry, and/or infrared spectroscopy.

Abbreviations as used herein, are defined as follows: “1 x” for once, “2 x” for twice, “3 x” for thrice, “° C.” for degrees Celsius, “eq” for equivalent or equivalents, “g” for gram or grams, “mg” for milligram or milligrams, “kg” for kilogram or kilograms, “L” for liter or liters, “mL” for milliliter or milliliters, “μL” for microliter or microliters, “N” for normal, “M” for molar, “mmol” for millimole or millimoles, “min” for minute or minutes, “h” for hour or hours, “rpm” for revolutions per minute, “rt” for room temperature (e.g., about 20, 21, 22, 23, 24, 25° C.), “RT” for retention time, “RBF” for round bottom flask, “atm” for atmosphere, “psi” for pounds per square inch, “conc.” for concentrate, “wt.” for weight, “sat” or “sat'd” for saturated, “SFC” for supercritical fluid chromatography “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “¹H” for proton, “δ” for delta, “s” for singlet, “d” for doublet, “t” for triplet, “q” for quartet, “m” for multiplet, “br” for broad, “Hz” for hertz, and “a”, “p”, “R”, “S”, “E”, and “Z” are stereochemical designations familiar to one skilled in the art.

The invention disclosed herein is also meant to encompass all pharmaceutically acceptable compounds of Formula I being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, 17O, ¹⁸O, ³¹P, ³²p, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labeled compounds of Formula I, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹C, ¹⁸F, ¹⁵O and ¹³N, can be useful in Positron Emission Topography (PET) studies for examining substrate receptor occupancy. Isotopically-labeled compounds of Formula I can generally be prepared by conventional techniques known to those skilled in the art or by processes analogous to those described in the Examples as set out below using an appropriate isotopically-labeled reagent in place of the non-labeled reagent previously employed.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

“Pharmaceutically effective amount” refers to an amount of the compound of the present disclosure in a formulation or combination thereof, that provides the desired therapeutic or pharmaceutical result.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

“Therapeutically effective amount” or “effective amount” as used herein refers to an amount that is effective to elicit the desired biological or medical response, including the amount of the compound that, when administered to a subject for treating a disease, is sufficient to effect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Subject” is any mammal, such as a mouse, a rat, a dog, a cat, including veterinary animals, such as a goat, a pig, a horse, a cow, or a donkey, and primates, such as non-human primates, e.g., a cynomolgous monkey, rhesus monkey, or chimpanzee, as well as humans. In some embodiments, the subject is a human.

### II. CRYSTALLINE FORMS

A crystalline form of the present disclosure can offer advantages to compounds and compositions described in the art, for example, greater convenience for use at large scale (e.g., freely flowing powders), retention of chemical and/or chiral purity over long-term storage, and/or lack of hygroscopicity.

In some embodiments, a crystalline form of the present disclosure is a crystalline form of a pharmaceutically acceptable salt or solvate of a compound of Formula (I):

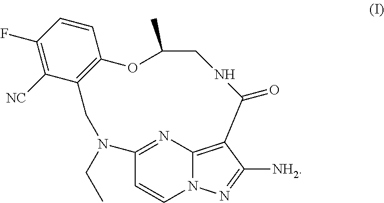 [SMILES: CCN1Cc2c(ccc(F)c2C#N)O[C@@H](C)CNC(=O)c2c(N)nn3ccc1nc23]

In some embodiments, a crystalline form of the present disclosure is a crystalline form of a 1, 5-naphthalenedisulfonate salt of a compound of Formula (I). In some embodiments, the crystalline form is a hemi-1, 5-naphthalendisulfonate salt. In some embodiments, the crystalline form is a hydrate.

In some embodiments, the crystalline form of a hemi-1, 5-naphthalendisulfonate salt of the compound of Formula (I) has an X-ray powder diffraction (XRPD) pattern having one or more, e.g., 2, 3, 4, 5, 6, 7, 8, 9, or 10 or more peaks at 6.8, 9.3, 10.2, 10.9, 12.8, 13.4, 14.5, 15.1, 16.7, 18.3, 18.7, 20.2, 21.6, 22.3, or 25.3 degrees 2θ (±0.2 degrees 2θ), the XRPD is made using CuK_{α1} radiation. In some embodiments, the crystalline form has an X-ray powder diffraction (XRPD) pattern having peaks at 10.2, 15.1, and 22.3 degrees 2θ (±0.2 degrees 2θ). In some embodiments, the crystalline form has an X-ray powder diffraction (XRPD) pattern further comprising 9.3 and 21.6 degrees 2θ (0.2 degrees 2θ). In some embodiments, the crystalline form has an X-ray powder diffraction (XRPD) pattern having peaks at 9.3, 10.2, 15.1, and 22.3 degrees 2θ (±0.2 degrees 2θ). In some embodiments, the crystalline form has an X-ray powder diffraction (XRPD) pattern having peaks at 9.3, 10.2, 15.1, 21.6, and 22.3 degrees 2θ (±0.2 degrees 2θ). In some embodiments, the crystalline form has an X-ray powder diffraction (XRPD) pattern having peaks at 6.8, 9.3, and 10.2 degrees 2θ (±0.2 degrees 2θ). In some embodiments, the crystalline form has an X-ray powder diffraction (XRPD) pattern having peaks at 6.8, 9.3, 10.2, and 10.9 degrees 2θ (±0.2 degrees 2θ). In some embodiments, the crystalline form has an X-ray powder diffraction (XRPD) pattern having peaks at 6.8, 9.3, 10.2, 10.9, and 12.8 degrees 2θ (±0.2 degrees 2θ). In some embodiments, the crystalline form has an X-ray powder diffraction (XRPD) pattern having peaks at 6.8, 9.3, 10.2, 10.9, 12.8, and 13.4 degrees 2θ (±0.2 degrees 2θ). In some embodiments, the crystalline form has an X-ray powder diffraction (XRPD) pattern having peaks at 6.8, 9.3, 10.2, 10.9, 12.8, 13.4, and 14.4 degrees 2θ (±0.2 degrees 2θ). In some embodiments, the crystalline form has an X-ray powder diffraction (XRPD) pattern having peaks at 6.8, 9.3, 10.2, 10.9, 12.8, 13.4, 14.4, and 15.1 degrees 2θ (±0.2 degrees 2θ). In some embodiments, the crystalline form has an X-ray powder diffraction (XRPD) pattern having peaks at 6.8, 9.3, 10.2, 10.9, 12.8, 13.4, 14.4, 15.1, 21.6, and 25.3 degrees 2θ (±0.2 degrees 2θ). In some embodiments, the crystalline form has an X-ray powder diffraction (XRPD) pattern having peaks as shown in Table 1.

TABLE 1
Naphthalenedisulfonate Form A

| Angle (degrees 2θ) | Relative Intensity (%) |
|---|---|
| 6.8 | 12.7 |
| 9.3 | 41.8 |
| 10.2 | 100.0 |
| 10.9 | 16.1 |
| 12.8 | 10.9 |
| 13.4 | 34.9 |
| 14.4 | 25.0 |
| 15.1 | 52.0 |
| 16.7 | 10.3 |
| 18.1 | 8.8 |
| 18.3 | 13.7 |
| 18.7 | 12.1 |
| 20.2 | 18.6 |
| 21.6 | 40.2 |
| 22.3 | 68.5 |
| 23.1 | 11.0 |
| 23.7 | 13.2 |
| 24.0 | 7.1 |
| 25.3 | 41.4 |
| 26.6 | 21.9 |
| 27.3 | 15.1 |
| 28.1 | 21.1 |

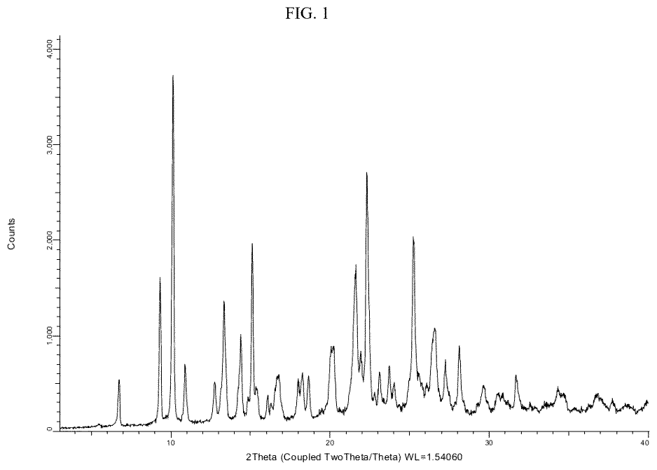

In some embodiments, the crystalline form has an X-ray powder diffraction (XRPD) pattern substantially as set forth in FIG. 1.

In some embodiments, the crystalline form has a differential scanning calorimetry (DSC) pattern having an endotherm at about 101° C. and/or about 227° C. In some embodiments, the crystalline form has a differential scanning calorimetry (DSC) pattern having an endotherm at about 101° C. and about 227° C.

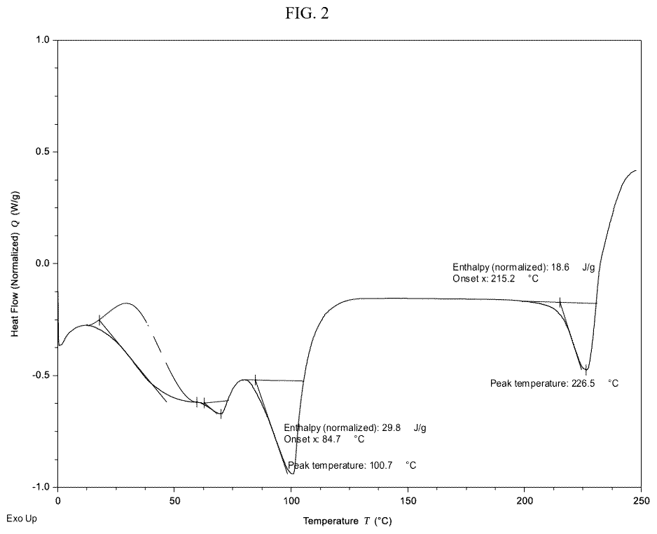

In some embodiments, the crystalline form has a differential scanning calorimetry (DSC) pattern substantially as set forth in FIG. 2.

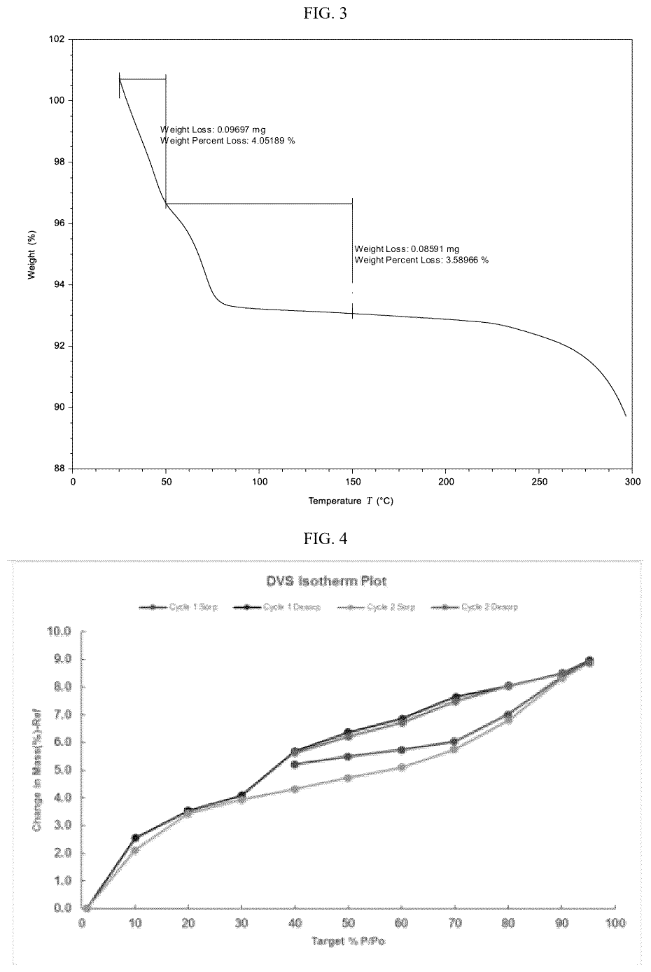

In some embodiments, the crystalline form has a thermogravimetric analysis (TGA) pattern substantially as set forth in FIG. 3.

In some embodiments, the crystalline form has a dynamic vapor sorption (DVS) isotherm substantially as set forth in FIG. 4.

In some embodiments, a crystalline form of the present disclosure is a crystalline form of a hemi-1, 5-naphthalenedisulfonate salt of a compound of Formula (I). In some embodiments, the crystalline form is a dichloromethane solvate.

In some embodiments, the crystalline form of a hemi-1, 5-naphthalenedisulfonate salt of a compound of Formula (I) dichloromethane solvate is characterized by a unit cell as determined by single crystal X-ray crystallography of the following dimensions: a=8.1400 (3) Å; b=11.5875 (5) Å; c=17.4332 (7) Å; α=82.064 (1°); β=84.129 (1°); and γ=81.812 (1°).

In some embodiments, the crystalline form of a hemi-1, 5-naphthalenedisulfonate salt of a compound of Formula (I) dichloromethane solvate has single crystal diffraction parameters according to Table 2.

TABLE 2
Single Crystal Data for Hemi-1,5-naphthalenedisulfonate
Salt Dichloromethane Solvate of Formula (I)

| | |
|---|---|
| C₁₀H₆O₆S₂•4(CH₂Cl₂)•2(C₂₀H₂₁FN₇O₂) | Z = 1 |
| Mᵣ = 1446.84 | F(000) = 744 |
| Triclinic, P1 | Dₓ = 1.496 Mg m⁻³ |
| a = 8.1400 (3) Å | Mo Kα radiation, |
| | λ = 0.71073 Å |
| b = 11.5875 (5) Å | Cell parameters from 5137 |
| | reflections |
| c = 17.4332 (7) Å | θ = 2.7°-26.1° |
| α = 82.064 (1)° | μ = 0.49 mm⁻¹ |
| β = 84.129 (1)° | T = 298 K |
| γ = 81.812 (1)° | Block, colorless |
| V = 1606.25 (11) Å³ | 0.15 × 0.08 × 0.05 mm |

In some embodiments, a crystalline form of the present disclosure is a crystalline form of an L-tartrate salt of a compound of Formula (I). In some embodiments, the L-tartaric acid salt of a compound of Formula (I) is a mono L-tartaric acid salt of the compound of Formula (I).

In some embodiments, the crystalline form of an L-tartrate salt of the compound of Formula (I) has an X-ray powder diffraction (XRPD) pattern having one or more, e.g., 2, 3, 4, 5, 6, 7, 8, 9, or 10 or more peaks at 7.1, 8.2, 10.5, 11.3, 12.0, 12.6, 13.1, 14.3, 14.6, 16.6, 18.8, 20.1, 21.1, 21.9, 22.5, 24.2, or 26.8 degrees 2θ (±0.2 degrees 2θ), wherein the XRPD is made using CuK_{α1} radiation. In some embodiments, the crystalline form has an X-ray powder diffraction (XRPD) pattern having peaks at 8.2, 10.5, and 12.6 degrees 2θ (±0.2 degrees 2θ). In some embodiments, the crystalline form has an X-ray powder diffraction (XRPD) pattern having peaks at 8.2, 10.5, 12.6, and 18.8 degrees 2θ (±0.2 degrees 2θ). In some embodiments, the crystalline form has an X-ray powder diffraction (XRPD) pattern having peaks at 7.1, 8.2, 10.5, 12.6, and 18.8 degrees 2θ (±0.2 degrees 2θ). In some embodiments, the crystalline form has an X-ray powder diffraction (XRPD) pattern having peaks at 7.1, 8.2, and 10.5 degrees 2θ (±0.2 degrees 2θ). In some embodiments, the crystalline form has an X-ray powder diffraction (XRPD) pattern having peaks at 7.1, 8.2, 10.5, and 11.3 degrees 2θ (±0.2 degrees 2θ). In some embodiments, the crystalline form has an X-ray powder diffraction (XRPD) pattern having peaks at 7.1, 8.2, 10.5, 11.3, and 12.0 degrees 2θ (0.2 degrees 2θ). In some embodiments, the crystalline form has an X-ray powder diffraction (XRPD) pattern having peaks at 7.1, 8.2, 10.5, 11.3, 12.0, and 12.6 degrees 2θ (0.2 degrees 2θ). In some embodiments, the crystalline form has an X-ray powder diffraction (XRPD) pattern having peaks at 7.1, 8.2, 10.5, 11.3, 12.0, 12.6, and 13.1 degrees 2θ (±0.2 degrees 2θ). In some embodiments, the crystalline form has an X-ray powder diffraction (XRPD) pattern having peaks at 7.1, 8.2, 10.5, 11.3, 12.0, 12.6, 13.1, and 14.3 degrees 2θ (0.2 degrees 2θ). In some embodiments, the crystalline form has an X-ray powder diffraction (XRPD) pattern having peaks at 7.1, 8.2, 10.5, 11.3, 12.0, 12.6, 13.1, 14.3, and 14.6 degrees 2θ (0.2 degrees 2θ). In some embodiments, the crystalline form has an X-ray powder diffraction (XRPD) pattern having peaks at 7.1, 8.2, 10.5, 11.3, 12.0, 12.6, 13.1, 14.3, 14.6, and 16.6 degrees 2θ (0.2 degrees 2θ). In some embodiments, the crystalline form has an X-ray powder diffraction (XRPD) pattern having peaks as shown in Table 3.

TABLE 3
L-Tartrate Form A

| Angle (degrees 2θ) | Relative Intensity (%) |
|---|---|
| 7.1 | 13.6 |
| 8.2 | 100.0 |
| 10.5 | 26.6 |
| 11.3 | 10.0 |
| 12.0 | 11.7 |
| 12.6 | 17.8 |
| 13.1 | 13.5 |
| 14.3 | 8.4 |
| 14.6 | 11.6 |
| 16.6 | 7.7 |
| 18.8 | 14.5 |
| 20.1 | 12.6 |
| 21.1 | 19.8 |
| 21.9 | 13.1 |
| 22.5 | 26.1 |
| 24.2 | 22.9 |
| 26.8 | 17.5 |

In some embodiments, the crystalline form has an X-ray powder diffraction (XRPD) pattern substantially as set forth in FIG. 5.

In some embodiments, the crystalline form has a differential scanning calorimetry (DSC) pattern having an endotherm at about 148° C.

In some embodiments, the crystalline form has a differential scanning calorimetry (DSC) pattern substantially as set forth in FIG. 6.

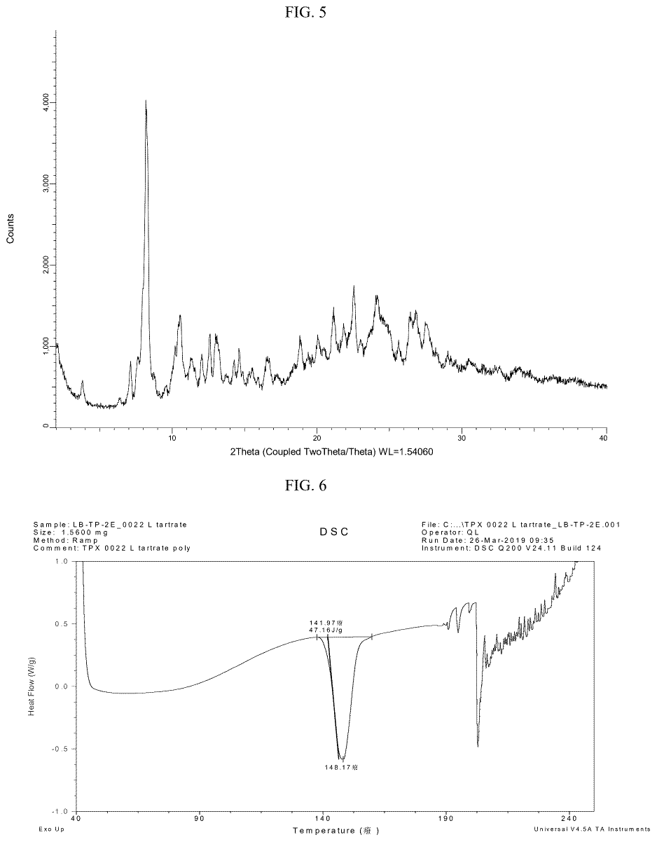

In some embodiments, the crystalline form has a thermogravimetric analysis (TGA) pattern substantially as set forth in FIG. 7.

In some embodiments, the crystalline form has an X-ray powder diffraction (XRPD) pattern substantially as set forth for the L-tartrate Form B in FIG. 8.

In some embodiments, the crystalline form has a differential scanning calorimetry (DSC) pattern having an endotherm at about 162° C. and/or about 189° C.

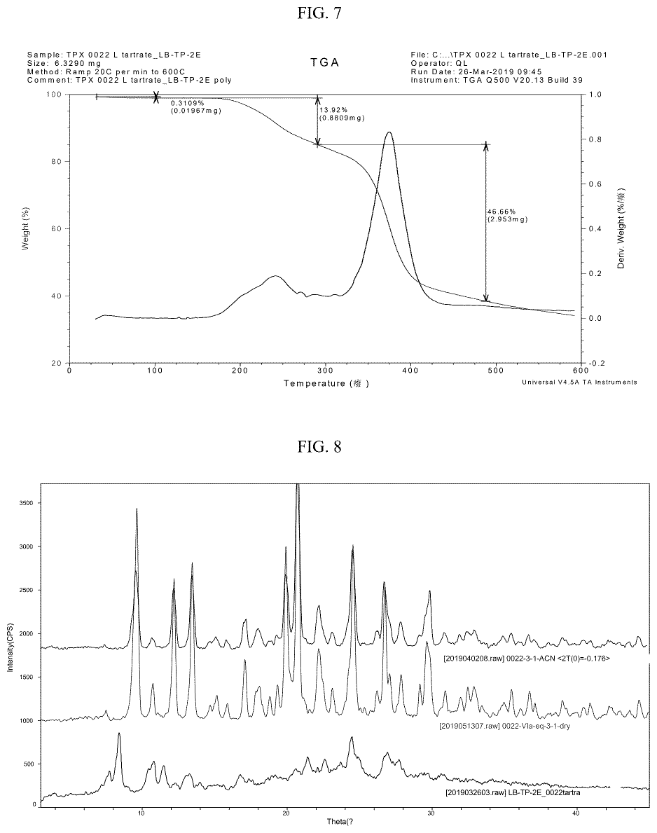

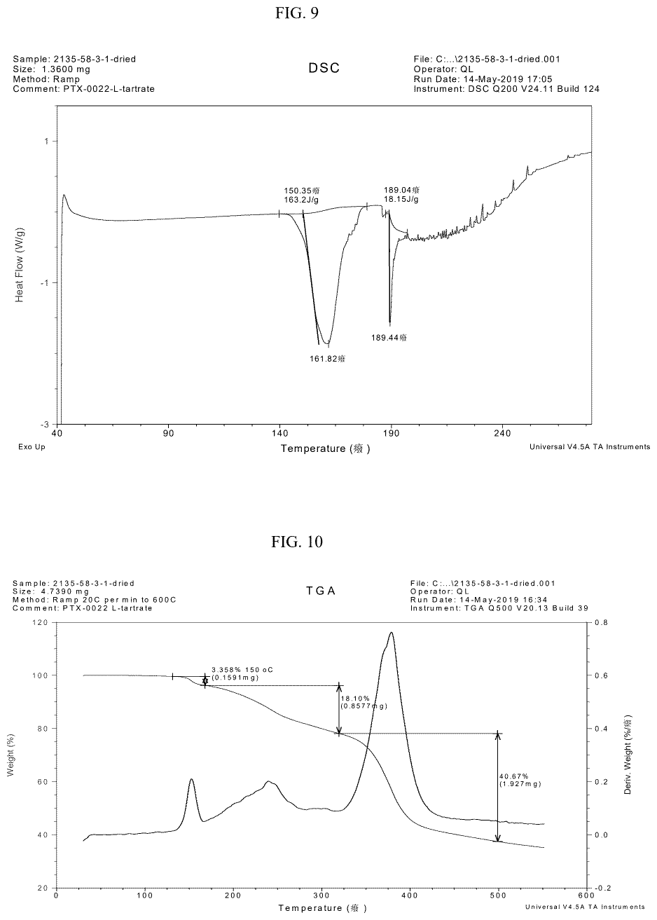

In some embodiments, the crystalline form has a differential scanning calorimetry (DSC) pattern substantially as set forth in FIG. 9.

In some embodiments, the crystalline form has a thermogravimetric analysis (TGA) pattern substantially as set forth in FIG. 10.

In some embodiments, the crystalline form has an X-ray powder diffraction (XRPD) pattern substantially as set forth for the L-tartrate Form C in FIG. 11.

In some embodiments, the crystalline form has a differential scanning calorimetry (DSC) pattern substantially as set forth in FIG. 12.

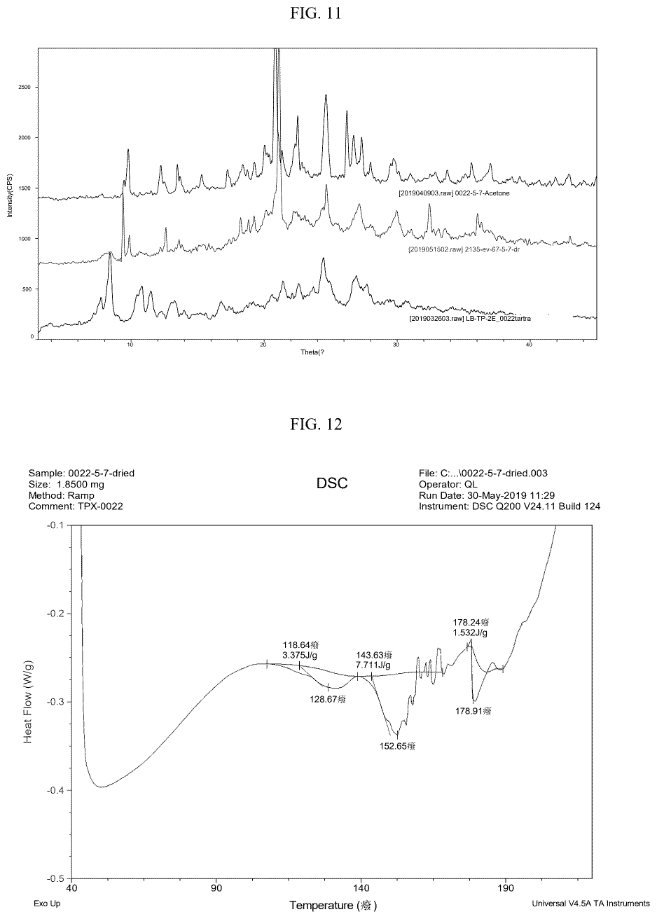

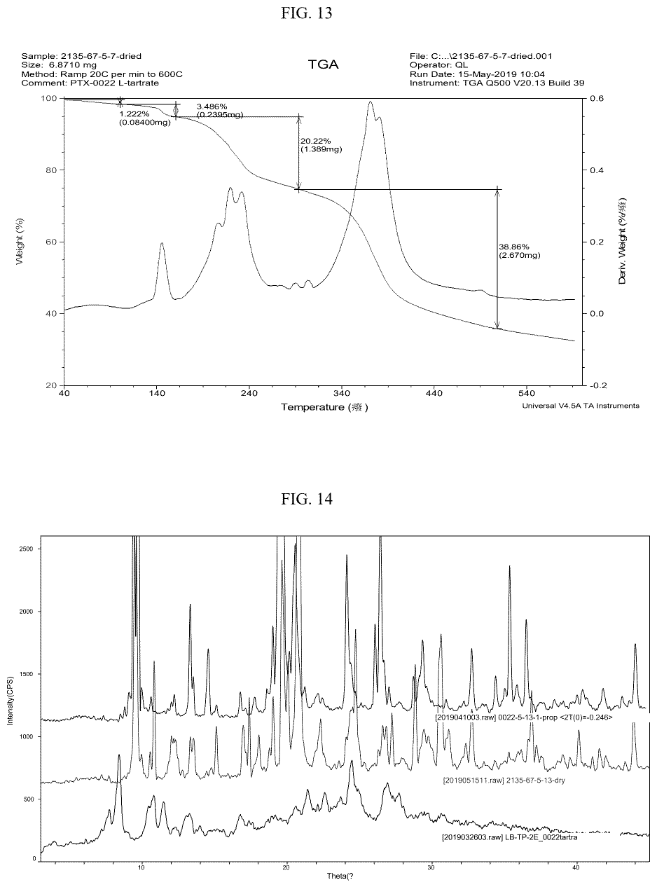

In some embodiments, the crystalline form has a thermogravimetric analysis (TGA) pattern substantially as set forth in FIG. 13.

In some embodiments, the crystalline form has an X-ray powder diffraction (XRPD) pattern substantially as set forth for the L-tartrate Form D in FIG. 14.

In some embodiments, the crystalline form has a differential scanning calorimetry (DSC) pattern having an endotherm at about 153° C.

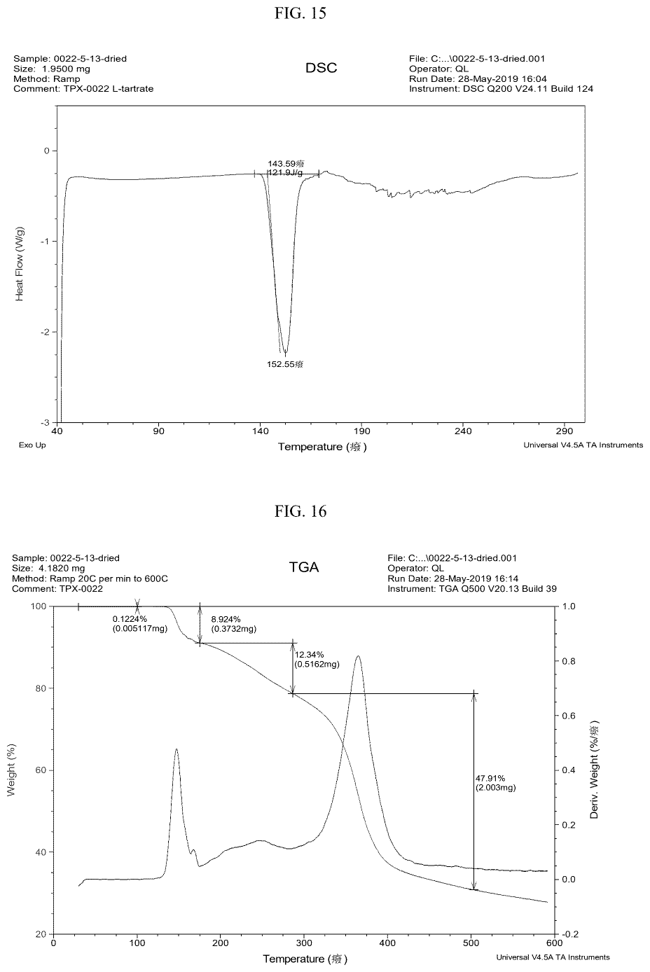

In some embodiments, the crystalline form has a differential scanning calorimetry (DSC) pattern substantially as set forth in FIG. 15.

In some embodiments, the crystalline form has a thermogravimetric analysis (TGA) pattern substantially as set forth in FIG. 16.

In some embodiments, the crystalline form has an X-ray powder diffraction (XRPD) pattern substantially as set forth for the L-tartrate Form E in FIG. 17.

In some embodiments, the crystalline form has a differential scanning calorimetry (DSC) pattern having an endotherm at about 162° C.

In some embodiments, the crystalline form has a differential scanning calorimetry (DSC) pattern substantially as set forth in FIG. 18.

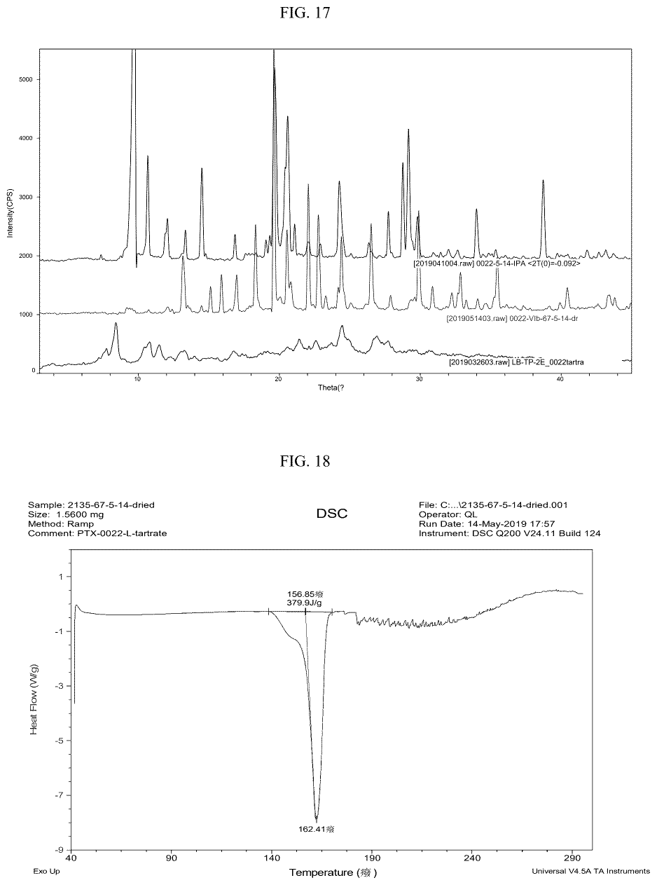

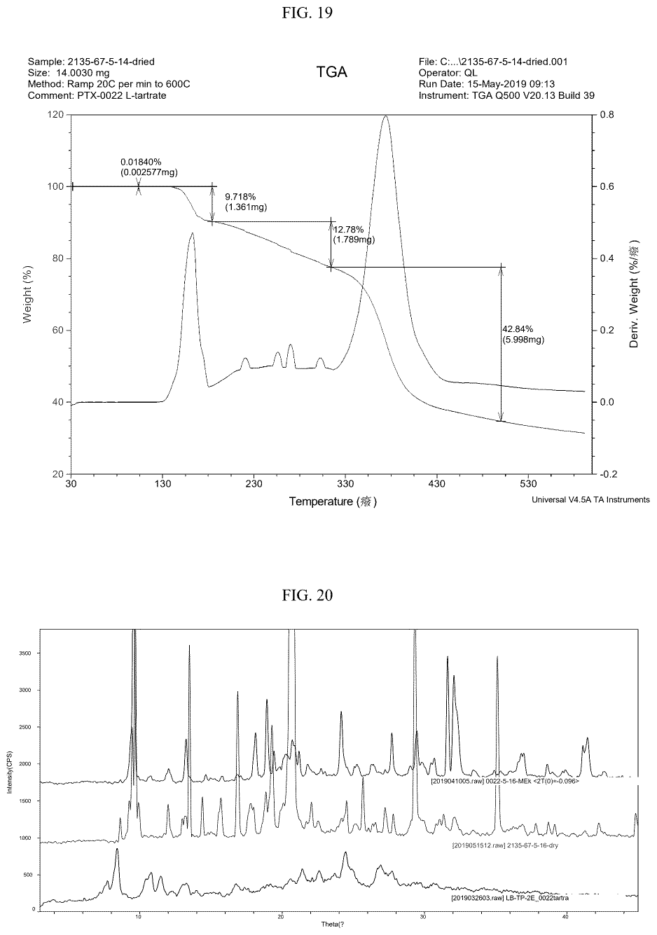

In some embodiments, the crystalline form has a thermogravimetric analysis (TGA) pattern substantially as set forth in FIG. 19.

In some embodiments, the crystalline form has an X-ray powder diffraction (XRPD) pattern substantially as set forth for the L-tartrate Form F in FIG. 20.

In some embodiments, the crystalline form has a differential scanning calorimetry (DSC) pattern having an endotherm at about 146° C.

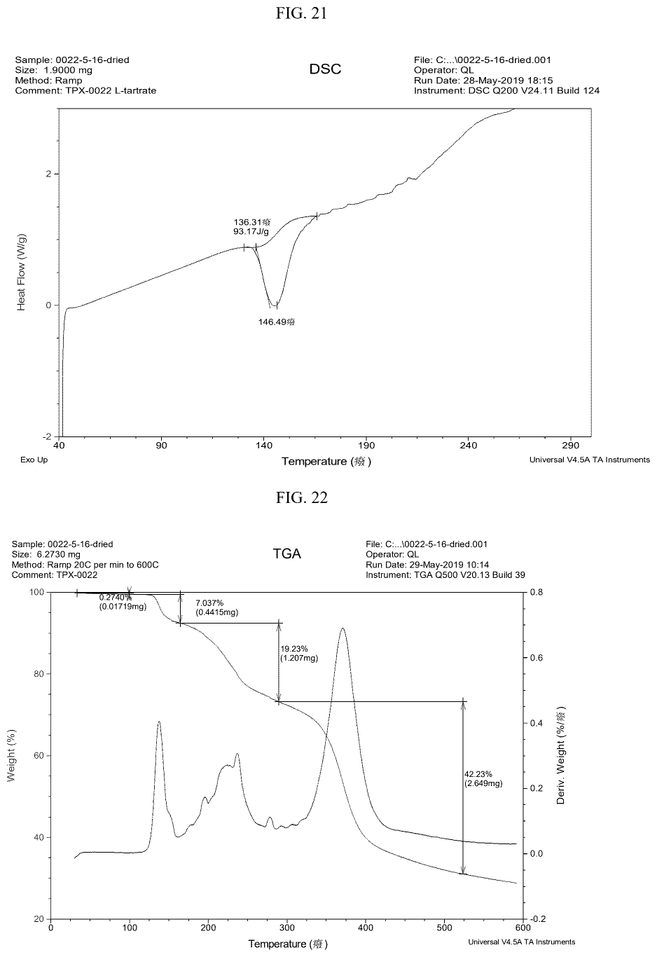

In some embodiments, the crystalline form has a differential scanning calorimetry (DSC) pattern substantially as set forth in FIG. 21.

In some embodiments, the crystalline form has a thermogravimetric analysis (TGA) pattern substantially as set forth in FIG. 22.

In some embodiments, a crystalline form of the present disclosure is a crystalline form of a hydrochloride salt of a compound of Formula (I). In some embodiments, the crystalline form has an X-ray powder diffraction (XRPD) pattern having peaks as shown in Table 4.

TABLE 4
Hydrochloride Salt

| 2θ (deg) | d-spacing (Å) | Relative intensity (a.u) |
|---|---|---|
| 4.70 | 18.78 | 12.69 |
| 7.33 | 12.05 | 43.47 |
| 8.72 | 10.13 | 99.65 |
| 9.41 | 9.39 | 100.00 |
| 9.99 | 8.85 | 2.54 |
| 10.52 | 8.40 | 16.60 |
| 10.96 | 8.07 | 4.60 |
| 11.89 | 7.44 | 11.19 |
| 13.14 | 6.73 | 8.44 |
| 15.18 | 5.83 | 4.48 |
| 15.90 | 5.57 | 9.53 |
| 16.99 | 5.22 | 1.39 |
| 17.98 | 4.93 | 9.51 |
| 18.67 | 4.75 | 5.69 |
| 19.75 | 4.49 | 27.20 |
| 20.28 | 4.37 | 16.09 |
| 21.95 | 4.05 | 10.76 |
| 22.51 | 3.95 | 4.94 |
| 22.99 | 3.87 | 3.44 |
| 23.63 | 3.76 | 19.50 |
| 24.22 | 3.67 | 19.89 |
| 24.80 | 3.59 | 12.97 |
| 26.15 | 3.41 | 13.61 |
| 26.61 | 3.35 | 1.56 |
| 28.38 | 3.14 | 4.00 |
| 28.85 | 3.09 | 12.69 |
| 29.38 | 3.04 | 5.28 |

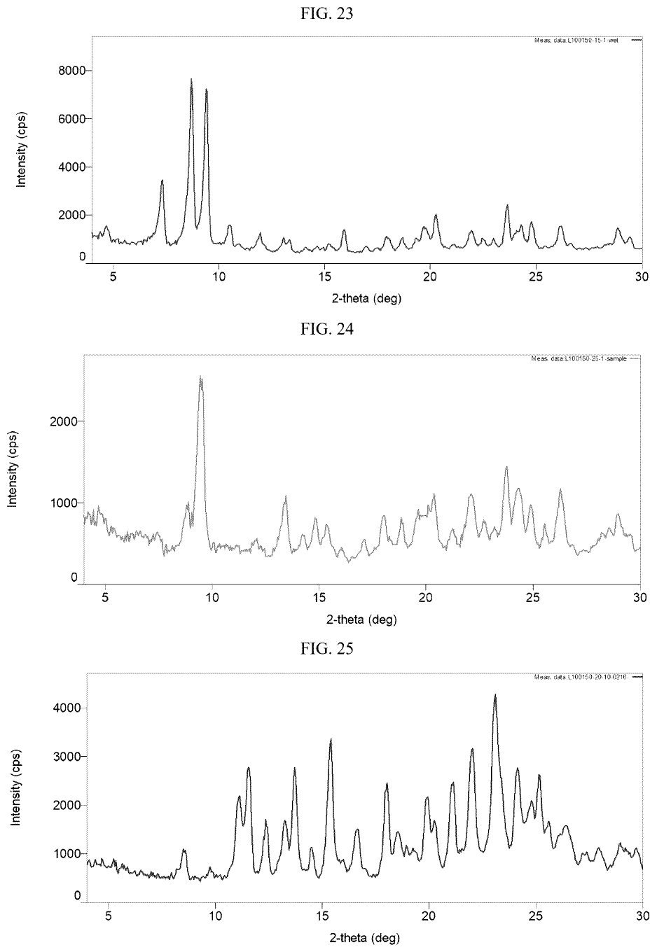

In some embodiments, the crystalline form has an X-ray powder diffraction (XRPD) pattern substantially as set forth in FIG. 23.

In some embodiments, a crystalline form of the present disclosure is a crystalline form of a sulfate salt of a compound of Formula (I). In some embodiments, the crystalline form has an X-ray powder diffraction (XRPD) pattern having peaks as shown in Table 5.

TABLE 5
Sulfate Salt Form A

| 2θ (deg) | d-spacing (Å) | Relative intensity (a.u) |
|---|---|---|
| 8.85 | 9.98 | 19.37 |
| 9.51 | 9.30 | 100.00 |
| 12.08 | 7.32 | 8.04 |
| 13.44 | 6.58 | 30.16 |
| 14.78 | 5.99 | 24.14 |
| 15.41 | 5.75 | 15.76 |
| 17.11 | 5.18 | 3.27 |
| 18.04 | 4.91 | 22.34 |
| 18.86 | 4.70 | 10.31 |
| 19.86 | 4.47 | 38.14 |
| 20.37 | 4.36 | 20.33 |
| 21.20 | 4.19 | 10.10 |
| 22.08 | 4.02 | 52.75 |
| 23.74 | 3.74 | 32.84 |
| 24.30 | 3.66 | 41.78 |
| 24.90 | 3.57 | 17.86 |
| 25.53 | 3.49 | 4.87 |
| 26.27 | 3.39 | 28.70 |
| 28.24 | 3.16 | 11.76 |
| 29.01 | 3.08 | 38.40 |

In some embodiments, the crystalline form has an X-ray powder diffraction (XRPD) pattern substantially as set forth in FIG. 24.

In some embodiments, a crystalline form of the present disclosure is a crystalline form of a sulfate salt of a compound of Formula (I). In some embodiments, the crystalline form has an X-ray powder diffraction (XRPD) pattern having peaks as shown in Table 6.

TABLE 6
Sulfate Salt Form B

| 2θ (deg) | d-spacing (Å) | Relative intensity (a.u) |
|---|---|---|
| 8.61 | 10.27 | 12.78 |
| 9.74 | 9.08 | 4.06 |
| 11.11 | 7.95 | 40.74 |
| 11.56 | 7.65 | 50.67 |
| 12.37 | 7.15 | 21.04 |
| 13.25 | 6.68 | 23.98 |
| 13.73 | 6.45 | 43.90 |
| 14.49 | 6.11 | 8.12 |
| 15.39 | 5.75 | 63.67 |
| 16.62 | 5.33 | 19.38 |
| 18.00 | 4.92 | 37.19 |
| 18.57 | 4.77 | 16.97 |
| 19.89 | 4.46 | 26.36 |
| 20.25 | 4.38 | 12.57 |
| 21.10 | 4.21 | 32.55 |
| 22.01 | 4.03 | 50.79 |
| 23.06 | 3.85 | 100.00 |
| 24.08 | 3.69 | 41.48 |
| 24.71 | 3.60 | 41.16 |
| 25.13 | 3.54 | 19.54 |
| 25.53 | 3.49 | 11.65 |
| 26.21 | 3.40 | 23.33 |
| 27.91 | 3.19 | 3.58 |
| 28.96 | 3.08 | 10.77 |
| 29.64 | 3.01 | 3.18 |

In some embodiments, the crystalline form has an X-ray powder diffraction (XRPD) pattern substantially as set forth in FIG. 25.

In some embodiments, a crystalline form of the present disclosure is a crystalline form of a solvate of a compound of Formula (I). In some embodiments, the solvate is a solvate of ethanol, methyl tert-butyl ether, heptane, cyclohexane, methylcyclohexane, ethyl acetate, isopropanol, dimethyl sulfoxide, toluene, methyl acetate, methyl isobutyl ketone, tetrahydrofuran, dichloromethane (DCM), acetonitrile, acetone, 2-methyltetrahydrofuran, methyl ethyl ketone, dimethylformamide, or N-methylpyrrolidone. In some embodiments, the solvate is a solvate of ethanol.

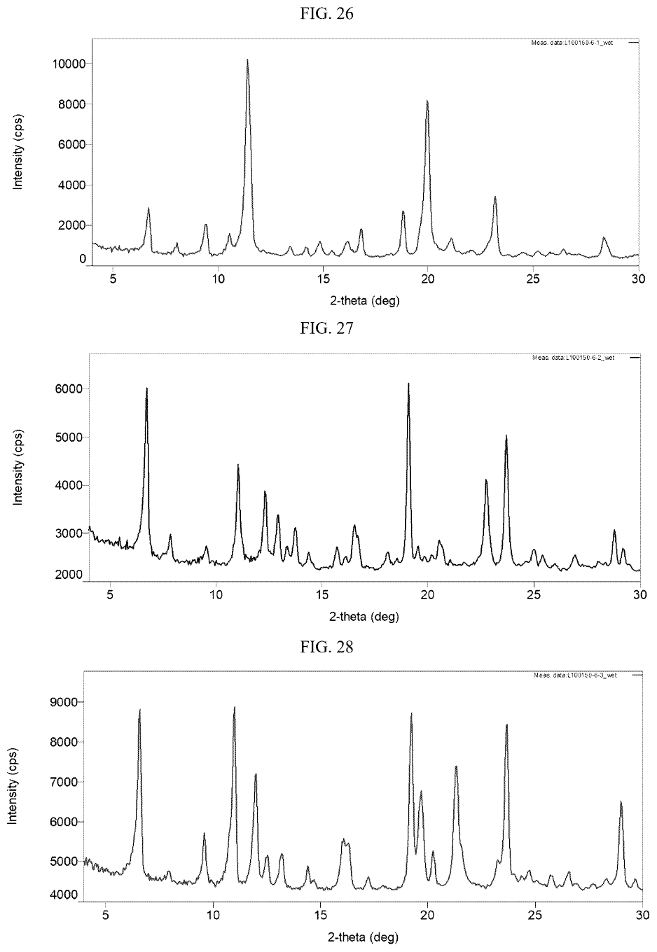

In some embodiments, a crystalline form of the present disclosure is a crystalline form of a methyl tert-butyl ether solvate of a compound of Formula (I). In some embodiments, the crystalline form has an X-ray powder diffraction (XRPD) pattern having peaks as shown in Table 7. In some embodiments, the crystalline form has an X-ray powder diffraction (XRPD) pattern substantially as set forth in FIG. 26.

TABLE 7
MTBE solvate

| 2θ (deg) | d-spacing (Å) | Relative intensity (a.u) |
|---|---|---|
| 6.70 | 13.18 | 14.75 |
| 8.04 | 10.98 | 2.07 |
| 9.38 | 9.42 | 10.09 |
| 10.57 | 8.37 | 5.37 |
| 11.40 | 7.76 | 100.00 |
| 13.42 | 6.59 | 4.30 |
| 14.17 | 6.25 | 2.84 |
| 14.82 | 5.97 | 6.23 |
| 15.39 | 5.75 | 1.84 |
| 16.13 | 5.49 | 7.82 |
| 16.79 | 5.28 | 9.56 |
| 18.81 | 4.71 | 13.28 |
| 19.92 | 4.45 | 77.96 |
| 21.11 | 4.21 | 9.72 |
| 22.05 | 4.03 | 2.51 |
| 23.17 | 3.84 | 24.65 |
| 24.48 | 3.63 | 1.09 |
| 25.20 | 3.53 | 1.36 |
| 26.25 | 3.39 | 3.31 |
| 26.42 | 3.37 | 1.09 |
| 28.37 | 3.14 | 7.50 |

In some embodiments, a crystalline form of the present disclosure is a crystalline form of a heptane solvate of a compound of Formula (I). In some embodiments, the crystalline form has an X-ray powder diffraction (XRPD) pattern having peaks as shown in Table 8. In some embodiments, the crystalline form has an X-ray powder diffraction (XRPD) pattern substantially as set forth in FIG. 27.

TABLE 8
Heptane solvate

| 2θ (deg) | d-spacing (Å) | Relative intensity (a.u) |
|---|---|---|
| 6.74 | 13.11 | 100.00 |
| 7.83 | 11.28 | 8.99 |
| 9.59 | 9.21 | 8.02 |
| 11.02 | 8.02 | 65.89 |
| 12.32 | 7.18 | 50.25 |
| 12.91 | 6.85 | 30.67 |
| 13.76 | 6.43 | 41.88 |
| 14.35 | 6.17 | 9.05 |
| 15.67 | 5.65 | 10.72 |
| 16.08 | 5.51 | 5.37 |
| 16.55 | 5.35 | 34.13 |
| 18.10 | 4.90 | 8.35 |
| 19.09 | 4.64 | 86.23 |
| 20.66 | 4.30 | 45.24 |
| 22.72 | 3.91 | 59.06 |
| 23.68 | 3.75 | 70.25 |
| 24.98 | 3.56 | 8.54 |
| 25.40 | 3.50 | 4.44 |
| 26.90 | 3.31 | 5.57 |
| 28.79 | 3.10 | 20.48 |
| 29.20 | 3.06 | 10.67 |
| 29.47 | 3.03 | 5.55 |

In some embodiments, a crystalline form of the present disclosure is a crystalline form of a cyclohexane solvate of a compound of Formula (I). In some embodiments, the crystalline form has an X-ray powder diffraction (XRPD) pattern having peaks as shown in Table 9. In some embodiments, the crystalline form has an X-ray powder diffraction (XRPD) pattern substantially as set forth in FIG. 28.

TABLE 9
Cyclohexane solvate

| 2θ (deg) | d-spacing (Å) | Relative intensity (a.u) |
|---|---|---|
| 6.58 | 13.42 | 96.42 |
| 7.97 | 11.09 | 5.17 |
| 9.58 | 9.23 | 21.78 |
| 10.98 | 8.06 | 100.00 |
| 11.98 | 7.38 | 65.81 |
| 12.52 | 7.06 | 19.14 |
| 13.21 | 6.70 | 23.22 |
| 14.39 | 6.15 | 16.33 |
| 16.21 | 5.46 | 48.86 |
| 17.24 | 5.14 | 5.69 |
| 19.24 | 4.61 | 78.52 |
| 19.69 | 4.50 | 63.27 |
| 20.26 | 4.38 | 15.16 |
| 21.32 | 4.16 | 95.65 |
| 23.31 | 3.81 | 17.90 |
| 23.67 | 3.76 | 76.02 |
| 24.71 | 3.60 | 20.39 |
| 25.71 | 3.46 | 4.46 |
| 26.54 | 3.36 | 6.39 |
| 26.90 | 3.31 | 1.21 |
| 28.32 | 3.15 | 3.34 |
| 28.99 | 3.08 | 48.70 |
| 29.68 | 3.01 | 3.75 |

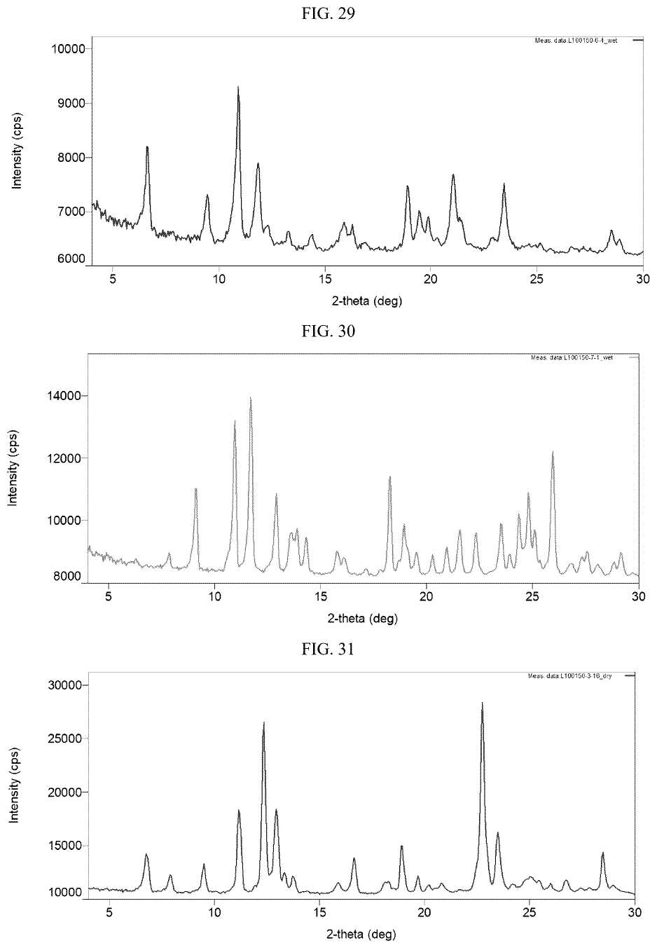

In some embodiments, a crystalline form of the present disclosure is a crystalline form of a methylcyclohexane solvate of a compound of Formula (I). In some embodiments, the crystalline form has an X-ray powder diffraction (XRPD) pattern having peaks as shown in Table 10. In some embodiments, the crystalline form has an X-ray powder diffraction (XRPD) pattern substantially as set forth in FIG. 29.

TABLE 10
Methylcyclohexane solvate

| 2θ (deg) | d-spacing (Å) | Relative intensity (a.u) |
|---|---|---|
| 6.61 | 13.35 | 45.28 |
| 9.45 | 9.35 | 20.63 |
| 10.89 | 8.12 | 100.00 |
| 11.81 | 7.49 | 80.74 |
| 13.25 | 6.67 | 9.71 |
| 14.37 | 6.16 | 8.50 |
| 15.90 | 5.57 | 37.21 |
| 16.28 | 5.44 | 5.06 |
| 18.91 | 4.69 | 36.14 |
| 19.47 | 4.56 | 25.72 |
| 19.87 | 4.46 | 16.42 |
| 20.28 | 4.38 | 6.12 |
| 21.06 | 4.22 | 77.80 |
| 22.95 | 3.87 | 6.77 |
| 23.47 | 3.79 | 39.95 |
| 28.51 | 3.13 | 21.55 |

In some embodiments, a crystalline form of the present disclosure is a crystalline form of an ethyl acetate solvate of a compound of Formula (I). In some embodiments, the crystalline form has an X-ray powder diffraction (XRPD) pattern having peaks as shown in Table 11. In some embodiments, the crystalline form has an X-ray powder diffraction (XRPD) pattern substantially as set forth in FIG. 30.

TABLE 11
Ethyl acetate solvate

| 2θ (deg) | d-spacing (Å) | Relative intensity (a.u) |
|---|---|---|
| 7.89 | 11.20 | 5.60 |
| 9.12 | 9.69 | 45.52 |
| 10.95 | 8.08 | 78.95 |
| 11.72 | 7.55 | 100.00 |
| 12.91 | 6.85 | 42.94 |
| 13.83 | 6.40 | 55.78 |
| 14.33 | 6.18 | 20.13 |
| 15.75 | 5.62 | 15.04 |
| 16.09 | 5.51 | 11.69 |
| 17.15 | 5.17 | 2.10 |
| 18.27 | 4.85 | 44.26 |
| 18.95 | 4.68 | 42.90 |
| 19.53 | 4.54 | 10.51 |
| 20.28 | 4.37 | 6.85 |
| 20.95 | 4.24 | 11.23 |
| 21.57 | 4.12 | 23.08 |
| 22.30 | 3.98 | 19.88 |
| 23.52 | 3.78 | 28.44 |
| 23.92 | 3.72 | 6.58 |
| 24.37 | 3.65 | 30.78 |
| 24.81 | 3.59 | 48.14 |
| 25.12 | 3.54 | 17.30 |
| 25.92 | 3.43 | 62.40 |
| 26.79 | 3.32 | 6.05 |
| 27.31 | 3.26 | 7.82 |
| 27.56 | 3.23 | 9.40 |
| 28.06 | 3.18 | 5.49 |
| 28.81 | 3.10 | 5.85 |
| 29.15 | 3.06 | 10.9 |

In some embodiments, a crystalline form of the present disclosure is a crystalline form of an ethanol solvate of a compound of Formula (I). In some embodiments, the crystalline form of an L-tartrate salt of the compound of Formula (I) has an X-ray powder diffraction (XRPD) pattern having one or more, e.g., 2, 3, 4, 5, 6, 7, 8, 9, or 10 or more peaks at 6.8, 7.9, 9.5, 11.2, 12.3, 12.9, 13.3, 13.7, 15.9, 16.6, 18.2, 18.9, 19.7, 20.8, 22.7, 23.5, 25.0, or 28.5 degrees 2θ (±0.2 degrees 2θ), wherein the XRPD is made using CuK_{α1} radiation. In some embodiments, the crystalline form has an X-ray powder diffraction (XRPD) pattern having peaks at 6.8, 9.5, and 11.2 degrees 2θ (0.2 degrees 2θ). In some embodiments, the crystalline form has an X-ray powder diffraction (XRPD) pattern having peaks at 6.8, 7.9, 9.5, and 11.2 degrees 2θ (0.2 degrees 2θ). In some embodiments, the crystalline form has an X-ray powder diffraction (XRPD) pattern having peaks at 6.8, 7.9, 9.5, 11.2, and 12.3 degrees 2θ (±0.2 degrees 2θ). In some embodiments, the crystalline form has an X-ray powder diffraction (XRPD) pattern having peaks at 6.8, 7.9, 9.5, 11.2, 12.3, and 12.9 degrees 2θ (±0.2 degrees 2θ). In some embodiments, the crystalline form has an X-ray powder diffraction (XRPD) pattern having peaks at 6.8, 7.9, 9.5, 11.2, 12.3, 12.9, and 22.7 degrees 2θ (±0.2 degrees 2θ). In some embodiments, the crystalline form has an X-ray powder diffraction (XRPD) pattern having peaks at 6.8, 7.9, 9.5, 11.2, 12.3, 12.9, 13.3, and 22.7 degrees 2θ (±0.2 degrees 2θ). In some embodiments, the crystalline form has an X-ray powder diffraction (XRPD) pattern having peaks at 6.8, 7.9, 9.5, 11.2, 12.3, 12.9, 13.3, 13.7, and 22.7 degrees 2θ (±0.2 degrees 2θ). In some embodiments, the crystalline form has an X-ray powder diffraction (XRPD) pattern having peaks at 6.8, 7.9, 9.5, 11.2, 12.3, 12.9, 13.3, 13.7, 15.9, and 22.7 degrees 2θ (±0.2 degrees 2θ). In some embodiments, the crystalline form has an X-ray powder diffraction (XRPD) pattern having peaks at 6.8, 7.9, 9.5, 11.2, 12.3, 12.9, 13.3, 13.7, 15.9, 16.6, and 22.7 degrees 2θ (±0.2 degrees 2θ).

In some embodiments, the crystalline form has an X-ray powder diffraction (XRPD) pattern having peaks as shown in Table 12. In some embodiments, the crystalline form has an X-ray powder diffraction (XRPD) pattern substantially as set forth in FIG. 31.

TABLE 12
Ethanol solvate

| Angle (degrees 2θ) | Relative Intensity (%) |
|---|---|
| 6.8 | 19.6 |
| 7.9 | 8.7 |
| 9.5 | 12.5 |
| 11.2 | 39.0 |
| 12.3 | 75.5 |
| 12.9 | 45.6 |
| 13.3 | 6.4 |
| 13.7 | 7.3 |
| 15.9 | 5.6 |
| 16.6 | 17.5 |
| 18.2 | 10.2 |
| 18.9 | 19.3 |
| 19.7 | 4.9 |
| 20.8 | 5.3 |
| 22.7 | 100.0 |
| 23.5 | 30.6 |
| 25.0 | 18.9 |
| 28.5 | 15.3 |

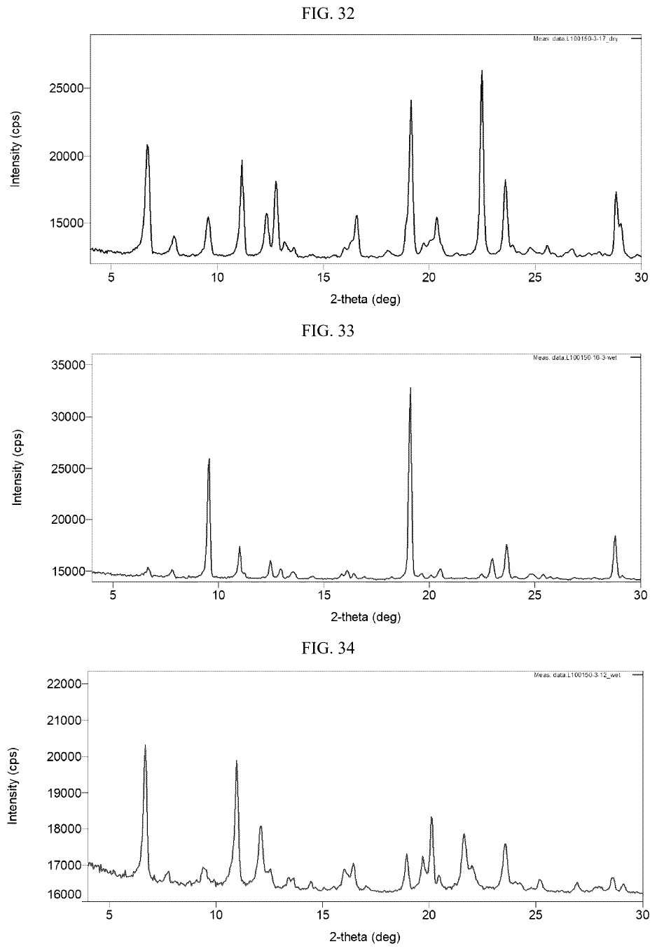

In some embodiments, a crystalline form of the present disclosure is a crystalline form of an isopropanol solvate of a compound of Formula (I). In some embodiments, the crystalline form has an X-ray powder diffraction (XRPD) pattern having peaks as shown in Table 13. In some embodiments, the crystalline form has an X-ray powder diffraction (XRPD) pattern substantially as set forth in FIG. 32.

TABLE 13
Isopropanol solvate.

| Angle (degrees 2θ) | Relative Intensity (%) |
|---|---|
| 6.72 | 81.60 |
| 7.94 | 15.59 |
| 9.55 | 29.57 |
| 11.15 | 58.26 |
| 12.30 | 28.09 |
| 12.75 | 44.63 |
| 13.18 | 13.73 |
| 13.59 | 6.57 |
| 16.57 | 40.48 |
| 19.12 | 90.44 |
| 19.84 | 14.49 |
| 20.35 | 34.27 |
| 22.47 | 100.00 |
| 23.57 | 53.31 |
| 24.76 | 8.12 |
| 28.78 | 27.98 |
| 29.01 | 16.31 |

In some embodiments, a crystalline form of the present disclosure is a crystalline form of a dimethylsulfoxide solvate of a compound of Formula (I). In some embodiments, the crystalline form has an X-ray powder diffraction (XRPD) pattern having peaks as shown in Table 14. In some embodiments, the crystalline form has an X-ray powder diffraction (XRPD) pattern substantially as set forth in FIG. 33.

TABLE 14
DMSO solvate

| 2θ (deg) | d-spacing (Å) | Relative intensity (a.u) |
|---|---|---|
| 6.66 | 13.26 | 6.09 |
| 7.81 | 11.32 | 4.63 |
| 9.53 | 9.28 | 62.21 |
| 10.98 | 8.05 | 21.77 |
| 12.48 | 7.09 | 11.43 |
| 12.95 | 6.83 | 6.19 |
| 13.55 | 6.53 | 7.73 |
| 14.46 | 6.12 | 3.24 |
| 15.82 | 5.60 | 3.21 |
| 16.09 | 5.50 | 5.92 |
| 16.42 | 5.39 | 3.52 |
| 16.91 | 5.24 | 1.90 |
| 18.24 | 4.86 | 0.66 |
| 19.08 | 4.65 | 100.00 |
| 19.61 | 4.52 | 3.32 |
| 20.08 | 4.42 | 2.06 |
| 20.50 | 4.33 | 7.63 |
| 22.47 | 3.95 | 2.62 |
| 22.97 | 3.87 | 13.07 |
| 23.66 | 3.76 | 21.57 |
| 24.07 | 3.69 | 1.19 |
| 24.84 | 3.58 | 5.11 |
| 25.38 | 3.51 | 2.31 |
| 25.71 | 3.46 | 1.01 |
| 27.80 | 3.21 | 0.67 |
| 28.78 | 3.10 | 26.39 |

In some embodiments, a crystalline form of the present disclosure is a crystalline form of a toluene solvate of a compound of Formula (I). In some embodiments, the crystalline form has an X-ray powder diffraction (XRPD) pattern having peaks as shown in Table 15. In some embodiments, the crystalline form has an X-ray powder diffraction (XRPD) pattern substantially as set forth in FIG. 34.

TABLE 15
Toluene solvate

| 2θ (deg) | d-spacing (Å) | Relative intensity (a.u) |
|---|---|---|
| 6.71 | 13.17 | 93.87 |
| 7.78 | 11.35 | 4.84 |
| 9.40 | 9.40 | 17.80 |
| 10.95 | 8.07 | 100.00 |
| 12.12 | 7.30 | 62.41 |
| 12.54 | 7.05 | 22.47 |
| 13.49 | 6.56 | 13.34 |
| 14.46 | 6.12 | 5.84 |
| 16.01 | 5.53 | 24.83 |
| 16.41 | 5.40 | 20.90 |
| 17.04 | 5.20 | 3.10 |
| 18.97 | 4.67 | 21.37 |
| 19.70 | 4.50 | 30.10 |
| 20.11 | 4.41 | 54.37 |
| 21.63 | 4.11 | 92.32 |
| 23.58 | 3.77 | 39.10 |
| 24.16 | 3.68 | 12.44 |
| 25.17 | 3.53 | 7.74 |
| 26.93 | 3.31 | 5.77 |
| 27.96 | 3.19 | 7.40 |
| 28.59 | 3.12 | 10.68 |
| 29.10 | 3.07 | 5.89 |

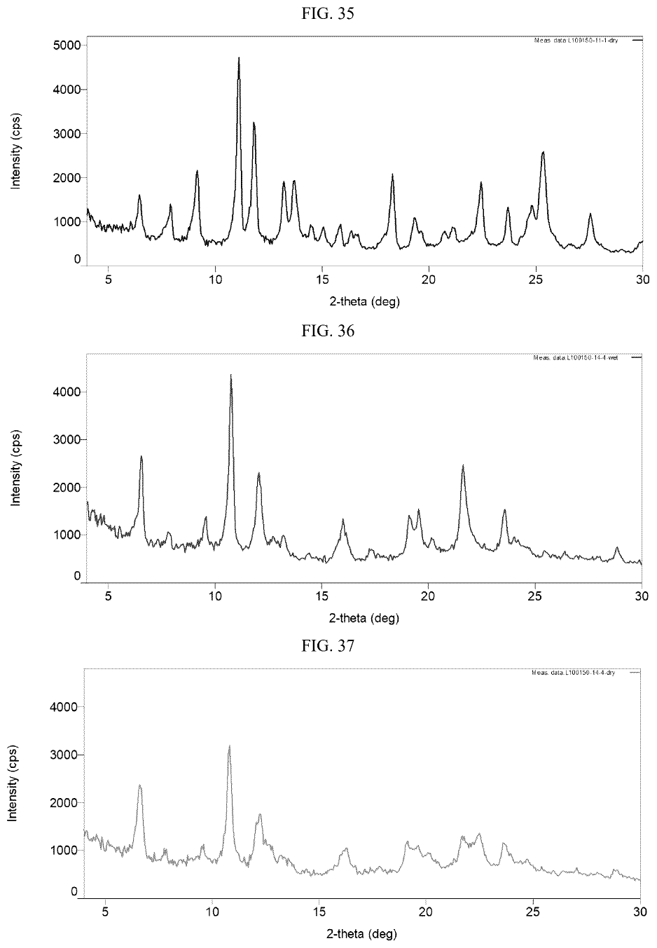

In some embodiments, a crystalline form of the present disclosure is a crystalline form of a methyl acetate solvate of a compound of Formula (I). In some embodiments, the crystalline form has an X-ray powder diffraction (XRPD) pattern having peaks as shown in Table 16. In some embodiments, the crystalline form has an X-ray powder diffraction (XRPD) pattern substantially as set forth in FIG. 35.

TABLE 16
Methyl acetate solvate

| 2θ (deg) | d-spacing (Å) | Relative intensity (a.u) |
|---|---|---|
| 6.47 | 13.66 | 33.93 |
| 7.94 | 11.12 | 22.12 |
| 9.12 | 9.69 | 49.90 |
| 11.12 | 7.95 | 100.00 |
| 11.81 | 7.49 | 79.91 |
| 13.19 | 6.71 | 41.39 |
| 13.68 | 6.47 | 47.97 |
| 14.48 | 6.11 | 7.32 |
| 15.03 | 5.89 | 7.73 |
| 15.80 | 5.60 | 9.26 |
| 16.48 | 5.37 | 11.53 |
| 17.99 | 4.93 | 11.31 |
| 18.31 | 4.84 | 37.08 |
| 19.31 | 4.59 | 27.05 |
| 21.13 | 4.20 | 25.90 |
| 22.46 | 3.95 | 60.49 |
| 23.68 | 3.75 | 22.58 |
| 24.79 | 3.59 | 55.19 |
| 25.34 | 3.51 | 89.46 |
| 27.53 | 3.24 | 37.27 |

In some embodiments, a crystalline form of the present disclosure is a crystalline form of a methyl isobutyl ketone solvate of a compound of Formula (I). In some embodiments, the crystalline form has an X-ray powder diffraction (XRPD) pattern having peaks as shown in Table 17. In some embodiments, the crystalline form has an X-ray powder diffraction (XRPD) pattern substantially as set forth in FIG. 36.

TABLE 17
Methyl isobutyl ketone solvate #1

| 2θ (deg) | d-spacing (Å) | Relative intensity (a.u) |
|---|---|---|
| 6.53 | 13.52 | 36.98 |
| 7.83 | 11.28 | 4.14 |
| 9.58 | 9.22 | 18.96 |
| 10.75 | 8.23 | 100.00 |
| 12.06 | 7.33 | 59.00 |
| 13.01 | 6.80 | 38.07 |
| 16.00 | 5.54 | 27.27 |
| 17.35 | 5.11 | 5.12 |
| 19.16 | 4.63 | 63.90 |
| 19.52 | 4.54 | 9.67 |
| 21.60 | 4.11 | 83.85 |
| 23.53 | 3.78 | 43.61 |
| 28.83 | 3.09 | 8.70 |

In some embodiments, a crystalline form of the present disclosure is a crystalline form of a methyl isobutyl ketone solvate of a compound of Formula (I). In some embodiments, the crystalline form has an X-ray powder diffraction (XRPD) pattern having peaks as shown in Table 18. In some embodiments, the crystalline form has an X-ray powder diffraction (XRPD) pattern substantially as set forth in FIG. 37.

TABLE 18
Methyl isobutyl ketone solvate #2

| 2θ (deg) | d-spacing (Å) | Relative intensity (a.u) |
|---|---|---|
| 6.63 | 13.31 | 47.75 |
| 9.57 | 9.24 | 20.78 |
| 10.78 | 8.20 | 100.00 |
| 12.19 | 7.25 | 93.46 |
| 13.28 | 6.66 | 9.47 |
| 16.20 | 5.47 | 26.28 |
| 19.35 | 4.58 | 70.69 |
| 21.76 | 4.08 | 61.12 |
| 22.44 | 3.96 | 23.68 |
| 23.64 | 3.76 | 27.62 |
| 24.49 | 3.63 | 12.12 |
| 28.79 | 3.10 | 7.78 |

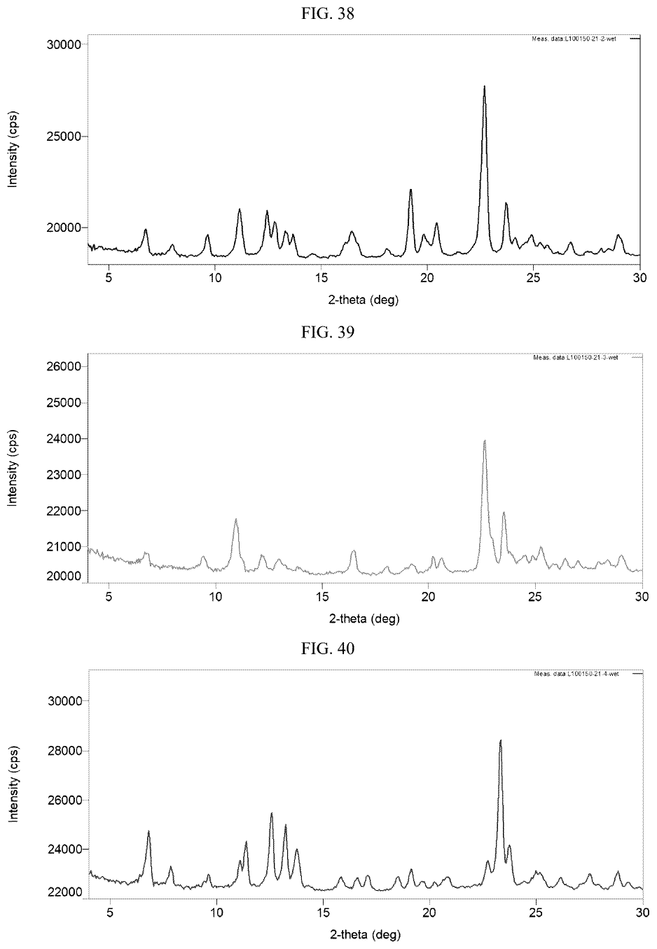

In some embodiments, a crystalline form of the present disclosure is a crystalline form of a tetrahydrofuran solvate of a compound of Formula (I). In some embodiments, the crystalline form has an X-ray powder diffraction (XRPD) pattern having peaks as shown in Table 19. In some embodiments, the crystalline form has an X-ray powder diffraction (XRPD) pattern substantially as set forth in FIG. 38.

TABLE 19
THF solvate

| 2θ (deg) | d-spacing (Å) | Relative intensity (a.u) |
|---|---|---|
| 6.73 | 13.12 | 14.59 |
| 7.99 | 11.05 | 6.47 |
| 9.64 | 9.16 | 10.37 |
| 11.16 | 7.93 | 28.73 |
| 12.44 | 7.11 | 22.74 |
| 12.81 | 6.91 | 18.49 |
| 13.31 | 6.64 | 14.58 |
| 13.67 | 6.47 | 11.92 |
| 14.59 | 6.07 | 1.70 |
| 15.47 | 5.72 | 0.63 |
| 16.47 | 5.38 | 28.36 |
| 18.07 | 4.91 | 6.18 |
| 19.20 | 4.62 | 31.12 |
| 19.86 | 4.47 | 15.78 |
| 20.42 | 4.35 | 16.38 |
| 22.70 | 3.91 | 100.00 |
| 23.69 | 3.75 | 27.46 |
| 24.87 | 3.58 | 25.96 |
| 26.76 | 3.33 | 4.13 |
| 28.26 | 3.16 | 5.80 |
| 28.95 | 3.08 | 13.39 |

In some embodiments, a crystalline form of the present disclosure is a crystalline form of a dichloromethane solvate of a compound of Formula (I). In some embodiments, the crystalline form has an X-ray powder diffraction (XRPD) pattern having peaks as shown in Table 20. In some embodiments, the crystalline form has an X-ray powder diffraction (XRPD) pattern substantially as set forth in FIG. 39.

TABLE 20
DCM solvate

| | | Relative |
|---|---|---|
| 2θ (deg) | d-spacing (Å) | intensity (a.u) |
| 6.82 | 12.95 | 6.22 |
| 9.40 | 9.40 | 10.56 |
| 10.95 | 8.07 | 46.93 |
| 12.15 | 7.28 | 13.99 |
| 12.95 | 6.83 | 18.42 |
| 13.85 | 6.39 | 5.92 |
| 16.49 | 5.37 | 18.04 |
| 18.05 | 4.91 | 2.88 |
| 19.26 | 4.61 | 6.27 |
| 20.20 | 4.39 | 5.31 |
| 20.58 | 4.31 | 8.29 |
| 22.64 | 3.92 | 100.00 |
| 23.50 | 3.78 | 33.44 |
| 24.50 | 3.63 | 6.22 |
| 25.27 | 3.52 | 24.12 |
| 26.38 | 3.38 | 4.93 |
| 26.99 | 3.30 | 4.32 |
| 28.29 | 3.15 | 9.93 |
| 29.04 | 3.07 | 8.94 |

In some embodiments, a crystalline form of the present disclosure is a crystalline form of an acetonitrile solvate of a compound of Formula (I). In some embodiments, the crystalline form has an X-ray powder diffraction (XRPD) pattern having peaks as shown in Table 21. In some embodiments, the crystalline form has an X-ray powder diffraction (XRPD) pattern substantially as set forth in FIG. 40.

TABLE 21
Acetonitrile solvate

| | | Relative |
|---|---|---|
| 2θ (deg) | d-spacing (Å) | intensity (a.u) |
| 6.80 | 12.99 | 37.84 |
| 7.86 | 11.24 | 13.11 |
| 9.65 | 9.16 | 9.05 |
| 11.08 | 7.98 | 11.15 |
| 11.39 | 7.76 | 35.86 |
| 12.57 | 7.03 | 52.42 |
| 13.22 | 6.69 | 42.99 |
| 13.77 | 6.43 | 40.39 |
| 14.32 | 6.18 | 3.97 |
| 15.81 | 5.60 | 10.34 |
| 16.57 | 5.35 | 8.19 |
| 17.08 | 5.19 | 9.91 |
| 18.54 | 4.78 | 8.03 |
| 19.15 | 4.63 | 10.86 |
| 19.68 | 4.51 | 5.46 |
| 20.26 | 4.38 | 2.78 |
| 20.82 | 4.26 | 10.34 |
| 22.72 | 3.91 | 14.48 |
| 23.31 | 3.81 | 100.00 |
| 23.73 | 3.75 | 33.80 |
| 24.38 | 3.65 | 2.96 |
| 25.06 | 3.55 | 23.34 |
| 26.14 | 3.41 | 6.64 |
| 27.14 | 3.28 | 1.83 |
| 27.49 | 3.24 | 8.28 |
| 27.88 | 3.20 | 1.41 |
| 28.81 | 3.10 | 9.96 |
| 29.31 | 3.04 | 3.23 |

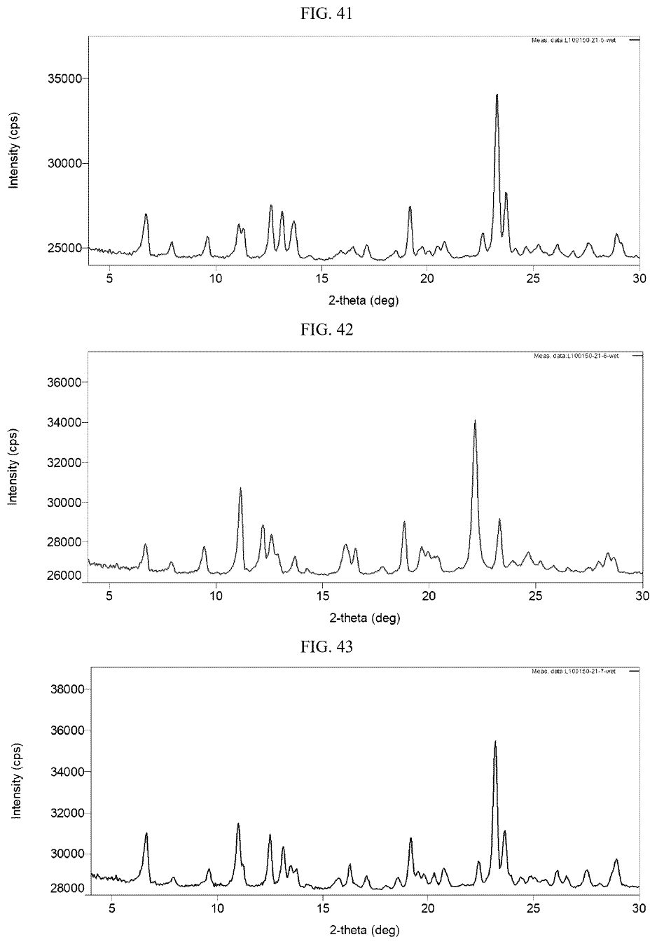

In some embodiments, a crystalline form of the present disclosure is a crystalline form of an acetone solvate of a compound of Formula (I). In some embodiments, the crystalline form has an X-ray powder diffraction (XRPD) pattern having peaks as shown in Table 22. In some embodiments, the crystalline form has an X-ray powder diffraction (XRPD) pattern substantially as set forth in FIG. 41.

TABLE 22
Acetone solvate

| | | Relative |
|---|---|---|
| 2θ (deg) | d-spacing (Å) | intensity (a.u) |
| 6.74 | 13.10 | 28.62 |
| 7.92 | 11.15 | 8.34 |
| 9.62 | 9.18 | 14.95 |
| 11.28 | 7.84 | 34.17 |
| 12.61 | 7.01 | 34.26 |
| 13.13 | 6.74 | 29.14 |
| 13.68 | 6.47 | 33.00 |
| 14.43 | 6.13 | 2.94 |
| 15.86 | 5.58 | 6.97 |
| 16.44 | 5.39 | 12.84 |
| 17.14 | 5.17 | 8.54 |
| 18.54 | 4.78 | 5.24 |
| 19.17 | 4.63 | 26.60 |
| 19.70 | 4.50 | 9.34 |
| 20.08 | 4.42 | 3.15 |
| 20.46 | 4.34 | 5.65 |
| 20.80 | 4.27 | 11.23 |
| 22.60 | 3.93 | 11.02 |
| 23.26 | 3.82 | 100.00 |
| 23.70 | 3.75 | 38.53 |
| 24.13 | 3.69 | 4.08 |
| 24.64 | 3.61 | 4.88 |
| 25.20 | 3.53 | 12.55 |
| 26.11 | 3.41 | 8.07 |
| 26.85 | 3.32 | 1.93 |
| 27.59 | 3.23 | 10.44 |
| 28.90 | 3.09 | 18.78 |

In some embodiments, a crystalline form of the present disclosure is a crystalline form of a 2-methyltetrahydrofuran solvate of a compound of Formula (I). In some embodiments, the crystalline form has an X-ray powder diffraction (XRPD) pattern having peaks as shown in Table 23. In some embodiments, the crystalline form has an X-ray powder diffraction (XRPD) pattern substantially as set forth in FIG. 42.

TABLE 23
2-Methyltetrahydrofuran solvate

| | | Relative |
|---|---|---|
| 2θ (deg) | d-spacing (Å) | intensity (a.u) |
| 6.68 | 13.23 | 13.37 |
| 7.90 | 11.18 | 5.02 |
| 9.46 | 9.34 | 13.70 |
| 11.17 | 7.92 | 44.79 |
| 12.16 | 7.27 | 17.90 |
| 12.57 | 7.04 | 45.32 |
| 13.66 | 6.48 | 10.50 |
| 14.25 | 6.21 | 4.51 |
| 16.08 | 5.51 | 24.08 |
| 16.54 | 5.35 | 10.98 |
| 17.84 | 4.97 | 3.86 |
| 18.56 | 4.78 | 1.68 |
| 18.82 | 4.71 | 20.71 |
| 19.76 | 4.49 | 22.38 |
| 20.31 | 4.37 | 10.82 |
| 22.15 | 4.01 | 100.00 |
| 23.30 | 3.82 | 22.60 |
| 23.94 | 3.71 | 6.98 |
| 24.64 | 3.61 | 16.06 |
| 25.21 | 3.53 | 5.40 |
| 25.82 | 3.45 | 2.33 |
| 26.47 | 3.37 | 0.95 |
| 27.47 | 3.24 | 1.97 |
| 27.91 | 3.19 | 4.18 |
| 28.43 | 3.14 | 16.84 |

In some embodiments, a crystalline form of the present disclosure is a crystalline form of a methyl ethyl ketone solvate of a compound of Formula (I). In some embodiments, the crystalline form has an X-ray powder diffraction (XRPD) pattern having peaks as shown in Table 24. In some embodiments, the crystalline form has an X-ray powder diffraction (XRPD) pattern substantially as set forth in FIG. 43.

TABLE 24
Methyl ethyl ketone solvate

| | | Relative |
|---|---|---|
| 2θ (deg) | d-spacing (Å) | intensity (a.u) |
| 6.65 | 13.29 | 40.05 |
| 7.90 | 11.18 | 5.51 |
| 9.58 | 9.22 | 15.14 |
| 10.94 | 8.08 | 57.26 |
| 12.49 | 7.08 | 33.06 |
| 13.11 | 6.75 | 28.63 |
| 13.48 | 6.56 | 13.99 |
| 13.73 | 6.44 | 12.24 |
| 15.73 | 5.63 | 11.57 |
| 16.29 | 5.44 | 14.03 |
| 17.08 | 5.19 | 6.77 |
| 18.56 | 4.78 | 5.86 |
| 19.16 | 4.63 | 28.86 |
| 19.49 | 4.55 | 9.42 |
| 19.79 | 4.48 | 8.84 |
| 20.26 | 4.38 | 7.49 |
| 20.75 | 4.28 | 13.83 |
| 22.40 | 3.97 | 11.78 |
| 23.17 | 3.84 | 100.00 |
| 23.63 | 3.76 | 37.94 |
| 24.38 | 3.65 | 3.68 |
| 24.89 | 3.58 | 8.45 |
| 25.48 | 3.49 | 3.42 |
| 26.08 | 3.41 | 8.55 |
| 26.52 | 3.36 | 4.56 |
| 27.51 | 3.24 | 10.71 |
| 28.12 | 3.17 | 1.06 |
| 28.92 | 3.09 | 24.32 |

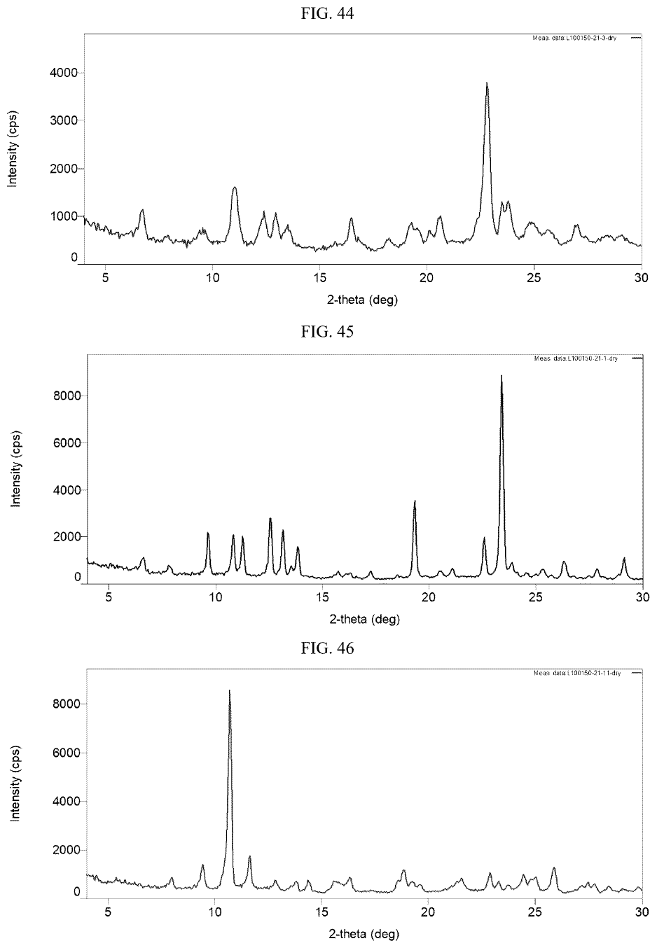

In some embodiments, a crystalline form of the present disclosure is a crystalline form of a dichloromethane solvate of a compound of Formula (I). In some embodiments, the crystalline form has an X-ray powder diffraction (XRPD) pattern having peaks as shown in Table 25. In some embodiments, the crystalline form has an X-ray powder diffraction (XRPD) pattern substantially as set forth in FIG. 44.

TABLE 25
Dichloromethane solvate

| | | Relative |
|---|---|---|
| 2θ (deg) | d-spacing (Å) | intensity (a.u) |
| 6.71 | 13.17 | 13.67 |
| 9.61 | 9.19 | 12.29 |
| 11.00 | 8.04 | 46.73 |
| 12.38 | 7.14 | 24.41 |
| 12.95 | 6.83 | 15.41 |
| 13.51 | 6.55 | 18.11 |
| 16.48 | 5.38 | 22.85 |
| 18.25 | 4.86 | 3.67 |
| 19.47 | 4.56 | 21.11 |
| 20.61 | 4.31 | 16.92 |
| 22.81 | 3.90 | 100.00 |
| 23.71 | 3.75 | 42.62 |
| 24.89 | 3.57 | 20.20 |
| 25.66 | 3.47 | 9.73 |
| 26.97 | 3.30 | 8.69 |

In some embodiments, a crystalline form of the present disclosure is a crystalline form of a dimethylformamide solvate of a compound of Formula (I). In some embodiments, the crystalline form has an X-ray powder diffraction (XRPD) pattern having peaks as shown in Table 26. In some embodiments, the crystalline form has an X-ray powder diffraction (XRPD) pattern substantially as set forth in FIG. 45.

TABLE 26
Dimethylformamide solvate

| | | Relative |
|---|---|---|
| 2θ (deg) | d-spacing (Å) | intensity (a.u) |
| 6.60 | 13.38 | 7.69 |
| 7.83 | 11.28 | 3.34 |
| 9.67 | 9.13 | 18.75 |
| 10.82 | 8.17 | 22.21 |
| 11.26 | 7.85 | 18.32 |
| 12.57 | 7.04 | 28.57 |
| 13.16 | 6.72 | 21.26 |
| 13.87 | 6.38 | 22.83 |
| 15.76 | 5.62 | 3.33 |
| 16.32 | 5.43 | 7.17 |
| 17.27 | 5.13 | 2.91 |
| 19.33 | 4.59 | 32.52 |
| 20.57 | 4.31 | 3.89 |
| 21.10 | 4.21 | 4.16 |
| 22.58 | 3.93 | 15.04 |
| 23.40 | 3.80 | 100.00 |
| 25.31 | 3.52 | 5.01 |
| 26.31 | 3.38 | 7.55 |
| 27.86 | 3.20 | 4.20 |
| 29.13 | 3.06 | 12.19 |

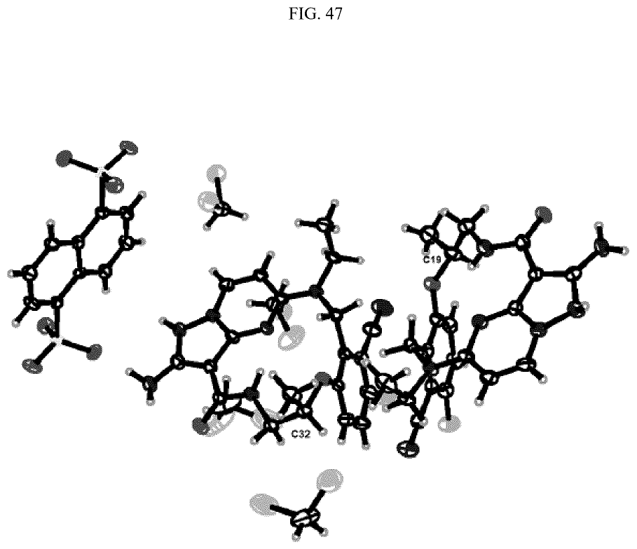

In some embodiments, a crystalline form of the present disclosure is a crystalline form of an N-methylpyrrolidone solvate of a compound of Formula (I). In some embodiments, the crystalline form has an X-ray powder diffraction (XRPD) pattern having peaks as shown in Table 27. In some embodiments, the crystalline form has an X-ray powder diffraction (XRPD) pattern substantially as set forth in FIG. 46.

TABLE 27
N-Methylpyrrolidone solvate

| | | Relative |
|---|---|---|
| 2θ (deg) | d-spacing (Å) | intensity (a.u) |
| 8.01 | 11.03 | 4.39 |
| 9.42 | 9.38 | 12.53 |
| 10.71 | 8.25 | 100.00 |
| 11.64 | 7.60 | 23.01 |
| 12.85 | 6.89 | 12.64 |
| 13.81 | 6.41 | 9.67 |
| 14.35 | 6.17 | 7.11 |
| 15.69 | 5.64 | 17.69 |
| 16.28 | 5.44 | 6.92 |
| 18.83 | 4.71 | 29.68 |
| 21.55 | 4.12 | 14.91 |
| 22.88 | 3.88 | 8.55 |
| 23.27 | 3.82 | 4.51 |
| 23.73 | 3.75 | 2.56 |
| 24.72 | 3.60 | 22.56 |
| 25.86 | 3.44 | 13.20 |
| 27.45 | 3.25 | 9.40 |
| 28.44 | 3.14 | 2.47 |
| 29.89 | 2.99 | 6.22 |

### III. PROCESSES

In some embodiments, the process of the present disclosure is a process for preparing a crystalline form of a hemi 1, 5-naplithalenedisulfonic acid salt of the compound of Formula (I) hydrate. In some embodiments, the process comprises: (a) combining a compound of Formula (I), 1, 5-naphthalenedisulfonic acid, and an alcohol to form a mixture; and (b) cooling the mixture; thereby preparing the crystalline form.

In some embodiments, the alcohol is methanol, ethanol, or isopropanol. In some embodiments, the alcohol is methanol. In some embodiments, the alcohol is ethanol. In some embodiments, the alcohol is isopropanol.

In some embodiments, the process comprises cooling the mixture to a temperature of from about 0° C. to about −40′° C. In some embodiments, the process comprises cooling the mixture to a temperature of about 0° C., about −10° C., about −20° C., about −30° C., or about −40° C.

In some embodiments, the process of the present disclosure is a process for preparing a crystalline form of an L-tartrate salt of the compound of Formula (I). In some embodiments, the process comprises combining a compound of Formula (I), L-tartaric acid, and an alcohol to form a mixture; thereby preparing the crystalline form.

In some embodiments, the alcohol is methanol, ethanol, or isopropanol. In some embodiments, the alcohol is methanol. In some embodiments, the alcohol is ethanol. In some embodiments, the alcohol is isopropanol.

In some embodiments, the process of the present disclosure is a process for preparing a crystalline form of a hemi 1, 5-naphthalenedisulfonic acid salt of the compound of Formula (I) dichloromethane solvate. In some embodiments, the process comprises combining a 1, 5-naphthalenedisulfonic acid salt of a compound of Formula (I) and dichloromethane, thereby preparing the crystalline form.

In some embodiments, the process of the present disclosure is a process for preparing a crystalline form of a solvate of the compound of Formula (I) by slurrying the compound with an appropriate solvent. In some embodiments, the process comprises combining a compound of Formula (I) and a solvent, thereby preparing the crystalline form of the solvate of the compound of Formula (I). In some embodiments, the solvent is MTBE, heptane, cyclohexane, methylcyclohexane, ethyl acetate, ethanol, isopropanol, toluene, methyl acetate, DMF, THF, dichloromethane, acetonitrile, acetone, 2-methyltetrahydrofuran, methyl ethyl ketone, or NMP. In some embodiments, the solvent is ethanol.

In some embodiments, the process comprises combining the compound of Formula (I) and from about 5 to about 100 volumes, such as from about 10 to about 50, from about 10 to about 40, or from about 20 to about 50 volumes of solvent to prepare the crystalline form of the solvate of the compound of Formula (I). In some embodiments, the process comprises combining the compound of Formula (I) and about 5, about 10, about 20, about 30, about 40, about 50, about 60, about 70, about 80, about 90, or about 100 volumes of solvent to prepare the crystalline form of the solvate of the compound of Formula (I).

### IV. COMPOSITIONS

In some embodiments, the present disclosure provides a composition comprising a therapeutically effective amount of the crystalline form of the compound of the present disclosure or a pharmaceutically acceptable salt thereof, and a pharmaceutically acceptable carrier or excipient. Also provided herein is a pharmaceutical composition comprising a therapeutically effective amount of the crystalline form of the compound of Formula I or a pharmaceutically acceptable salt thereof, and a pharmaceutically acceptable carrier or excipient.

The crystalline form of the compound of Formula I described herein is formulated with conventional carriers and excipients, which will be selected in accord with conventional practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, but is ordinarily about 7 to 10.

While it is possible for the active ingredient to be administered alone it may be preferable to present them as pharmaceutical compositions. The compositions, both for veterinary and for human use, comprise the active ingredient, as above defined, together with one or more acceptable carriers and optionally other therapeutic ingredients, particularly those additional therapeutic ingredients as discussed herein. The carrier(s) must be “acceptable” in the sense of being compatible with the other ingredients of the formulation and physiologically innocuous to the recipient thereof.

The compositions may conveniently be presented in unit dosage form and may be prepared by any of the methods well known in the art of pharmacy. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general the compositions are prepared by uniformly and intimately bringing into association the active ingredient with liquid carriers or finely divided solid carriers or both, and then, if necessary, shaping the product.

Compositions suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Pharmaceutical compositions herein comprise the active ingredient together with one or more pharmaceutically acceptable carriers or excipients and optionally other therapeutic agents. Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, solutions, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Compositions for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions may be in the form of a sterile injectable or intravenous preparations, such as a sterile injectable aqueous or oleaginous suspension. This suspension may be formulated according to the known art using those suitable dispersing or wetting agents and suspending agents which have been mentioned above. The sterile injectable or intravenous preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compound with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The compositions are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Exemplary unit dosage formulations are those containing a daily dose or unit daily sub-dose, as herein above recited, or an appropriate fraction thereof, of the active ingredient.

It should be understood that in addition to the ingredients particularly mentioned above the formulations may include other agents conventional in the art having regard to the type of composition in question, for example those suitable for oral administration may include flavoring agents.

Further provided are veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Effective dose of active ingredient depends at least on the nature of the condition being treated, toxicity, whether the crystalline form is being used prophylactically (lower doses) or against an active disease, the method of delivery, and the pharmaceutical formulation, and will be determined by the clinician using conventional dose escalation studies. It can be expected to be from about 0.0001 to about 100 mg/kg body weight per day; for example, from about 0.01 to about 10 mg/kg body weight per day. In some embodiments, the effective dose is from about 0.01 to about 5 mg/kg body weight per day; for example typically, from about 0.05 to about 0.5 mg/kg body weight per day. For example, the daily candidate dose for an adult human of approximately 70 kg body weight will range from 1 mg to 1000 mg, for example between 5 mg and 500 mg, and may take the form of single or multiple doses.

### V. ROUTES OF ADMINISTRATION

The crystalline form of the compound of Formula I or pharmaceutically acceptable salt thereof (also referred to herein as the active ingredient), can be administered by any appropriate route appropriate. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with for example the condition of the recipient.

The crystalline form of the compound of the present disclosure may be administered to an individual in accordance with an effective dosing regimen for a desired period of time or duration, such as at least about one week, at least about two weeks, at least about three weeks, one month, at least about 2 months, at least about 3 months, at least about 6 months, or at least about 12 months or longer. In one variation, the compound is administered on a daily or intermittent schedule for the required duration, up to the individual's life.

The dosage or dosing frequency of the crystalline form of the compound of the present disclosure may be adjusted over the course of the treatment, based on the judgment of the administering physician.

The crystalline form of the compound may be administered to an individual (e.g., a human) in an effective amount. In some embodiments, the crystalline form of the compound is administered once daily.

The crystalline form of the compound can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

The crystalline form of the compound of the present disclosure may be combined with one or more additional therapeutic agents in any dosage amount of the compound of the present disclosure (e.g., from 1 mg to 1000 mg of compound). Therapeutically effective amounts may include from about 1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose. Other therapeutically effective amounts of the compound of the present disclosure are about 100, 125, 150, 175, 200, 225, 250, 275, 300, 325, 350, 375, 400, 425, 450, 475, or about 500 mg per dose. Other therapeutically effective amounts of the compound of the present disclosure are about 100 mg per dose, or about 125, 150, 175, 200, 225, 250, 275, 300, 350, 400, 450, or about 500 mg per dose. A single dose can be administered hourly, daily, or weekly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In some embodiments, a single dose can be administered once every week. A single dose can also be administered once every month.

The frequency of dosage of the crystalline form of the compound of the present disclosure are determined by the needs of the individual patient and can be, for example, once per day or twice, or more times, per day. Administration of the compound continues for as long as necessary to treat the viral infection. For example, the compound can be administered to a human being infected with a virus for a period of from 20 days to 180 days or, for example, for a period of from 20 days to 90 days or, for example, for a period of from 30 days to 60 days.

Administration can be intermittent, with a period of several or more days during which a patient receives a daily dose of the compound of the present disclosure followed by a period of several or more days during which a patient does not receive a daily dose of the compound. For example, a patient can receive a dose of the compound every other day, or three times per week. Again by way of example, a patient can receive a dose of the compound each day for a period of from 1 to 14 days, followed by a period of 7 to 21 days during which the patient does not receive a dose of the compound, followed by a subsequent period (e.g., from 1 to 14 days) during which the patient again receives a daily dose of the compound. Alternating periods of administration of the compound, followed by non-administration of the compound, can be repeated as clinically required to treat the patient.

In one embodiment, pharmaceutical compositions comprising the crystalline form of the compound of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In one embodiment, kits comprising the crystalline form of the compound of the present disclosure, or a pharmaceutically acceptable salt thereof, in combination with one or more (e.g., one, two, three, four, one or two, one to three, or one to four) additional therapeutic agents are provided.

### VI. METHODS AND/OR USES

In some embodiments, a method of the present disclosure is a method of treating a cancer in a subject in need thereof, comprising administering to the subject a therapeutically effective amount of a crystalline form of a compound of Formula I or pharmaceutically acceptable salt thereof.

In some embodiments, a use of the present disclosure is a use in the manufacture of a medicament for treating a cancer in a subject in need thereof, comprising a therapeutically effective amount of a crystalline form of a compound of Formula I or pharmaceutically acceptable salt thereof.

In some embodiments, a therapeutically effective amount of a crystalline form of a compound of Formula I or pharmaceutically acceptable salt thereof is for use in treating a cancer in a subject in need thereof.

In some embodiments, the subject has received prior treatment with one or more therapeutic agents.

In some embodiments, the cancer is a MET-driven solid tumor. In some embodiments, the cancer is a MET-driven advanced solid tumor.

In some embodiments, the cancer is mediated by a genetically altered MET. In some embodiments, the genetically altered MET gene comprises a point mutation that is expressed in the c-Met protein. In some embodiments, the genetically altered MET comprises a point mutation expressed in the c-Met protein at one or more of positions P991, T992, D1010, V1092, H1094, G1163, T1173, H1094, N1100, Y1003, H1106, V1070, V1188, V1092, H1094, G1162, L1195, F1200, V1220, D1228, Y1230, D1231, Y1235, D1246, Y1248, M1250, and M1268. In some embodiments, the genetically altered MET comprises a point mutation expressed in the c-Met protein that is selected from the group consisting of P991S, T992I, D1010H, D1010Y, V1092I, H1094N, H1094R, H1094Y, N1100K, N1100S, Y1003C, Y1003F, Y1003H, H1106D, V1070A, V1092I, V1188I, T1173I, H1094Y, G1163R, L1195F, F1195I, L1195V, F1200I, V1220I, D1228N, D1228H, D1228V, Y1230A, Y1230C, Y1230D, Y1230H, Y1230H, Y1230S, D1231Y, Y1235D, D1246N, D1246H, Y1248D, Y1248H, Y1248C, M1250T, and M1268T. In some embodiments, the genetically altered MET comprises a point mutation expressed in the c-Met protein that is selected from the group consisting of T1173I, P991S, M1250T, T992I, V1092I, F1200I, Y1235D, Y1230H, D1246N, D1246H, Y1248D, Y1248H, Y1248C, and M1268T.

In some embodiments, the cancer is a carcinoma, a sarcoma, a lymphoma, Hodgekin's disease, a melanoma, a mesothelioma, Burkitt's lymphoma, a nasopharyngeal carcinoma, a leukemia, a lung cancer, a breast cancer, a hereditary human papillary renal carcinoma, a sporadic human papillary renal carcinoma, a childhood hepatocellular carcinoma, or a myeloma. In some embodiments, the cancer is selected from the group consisting of ALCL, NSCLC, neuroblastoma, inflammatory myofibroblastic tumor, renal cancer, adult renal cell carcinoma, pediatric renal cell carcinoma, breast cancer, triple negative breast cancer, triple positive breast cancer, HER⁺ breast cancer, mouth cancer, esophageal cancer, laryngeal cancer, pancreatic cancer, bladder cancer, colon cancer, colonic adenocarcinoma, glioblastoma, glioblastoma multiforme, thyroid cancer, anaplastic thyroid cancer, endocrine cancer, bone cancer, cholangiocarcinoma, ovarian cancer, cervical cancer, uterine cancer, testicular cancer, gastric cancer, gastric adenocarcinoma, colorectal cancer, rectal cancer, liver cancer, kidney cancer, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cancer, head and neck squamous cell carcinoma, pediatric glioma CML, prostate cancer, lung squamous carcinoma, ovarian serous cystadenocarcinoma, skin cutaneous melanoma, metastatic castration-resistant prostate cancer, Hodgkin lymphoma, neuroendocrine tumors, serous and clear cell endometrial cancer.

In some embodiments, the subject has been previously treated with a cancer therapeutic. In some embodiments, the subject has been previously treated with a cancer therapeutic, and the cancer has developed resistance to the cancer therapeutic. In some embodiments, the resistance is a primary intrinsic resistance. In some embodiments, the resistance is an acquired resistance from mutation(s). In some embodiments, the resistance is a bypass resistance. In some embodiments, the resistance is an EMT-based resistance.

In some embodiments, the cancer is exhibiting bypass resistance. In some embodiments, the bypass resistance is mediated by a SRC/CSF1R.

### VII. EXAMPLES

Exemplary methods of the description will now be described by reference to the specific examples that follow. Artisans will recognize that, to obtain the various compounds herein, starting materials may be suitably selected so that the ultimately desired substituents will be carried through the reaction scheme with or without protection as appropriate to yield the desired product.

### ABBREVIATIONS

| | |
|---|---|
| Å | Angstroms |
| ° C. | degrees Celsius |
| 2-Me-THF | 2-methyltetrahydrofuran |
| Boc | tert-butoxycarbonyl |
| Cbz | carboxybenzoyl |
| DCM | dichloromethane |
| DMF | dimethylformamide |
| eq | equivalents |
| FDPP | pentafluorophenyl diphenylphosphinate |
| g | grams |
| h | hours |
| HPLC | high performance liquid chromatography |
| kg | kilograms |
| min | minutes |
| mmol | millimoles |
| Ms | methanesulfonyl |
| MsCl | methanesulfonyl chloride |
| MTBE | methyl tert-butyl ether |
| TEA | triethylamine |
| TFA | trifluoroacetic acid |
| THF | tetrahydrofuran |

General methods include the following instruments and parameters.

X-ray Powder Diffractometer (XRPD)

| | |
|---|---|
| Instrument | Bruker D8 Advance |

Method 1

| | |
|---|---|
| X-ray geometry | Reflection |
| Detector | LYNXEYE_XE_T(1D mode) |
| Open angle | 2.94° |
| Radiation | Cu/K-Alpha1 (λ = 1.5406 Å) |
| X-ray generator power | 40 kV, 40 mA |
| Primary beam | Twin_Primary motorized slit |
| path slits | 10.0 mm by sample length; |
| | SollerMount axial soller 2.5° |
| Secondary beam | Detector OpticsMount soller |
| path slits | slit 2.5°; Twin_Secondary |
| | motorized slit 5.2 mm |
| Scan mode | Continuous scan |
| Scan type | Locked coupled |
| Step size | 0.02° |
| Time per step | 0.3 second per step |
| Scan range | 2° to 40° |
| Sample rotation speed | 15 rpm |
| Sample holder | Monocrystalline silicon, flat surface |

Method 2

| | |
|---|---|
| X-ray geometry | Reflection |
| Detector | LYNXEYE_XE_T(1D mode) |
| Open angle | 2.94° |
| Radiation | Cu/K-Alpha1 (λ = 1.5406 Å) |
| X-ray generator power | 40 kV, 40 mA |
| Primary beam | Twin_Primary motorized slit |
| path slits | 10.0 mm by sample length; |
| | SollerMount axial soller 2.5° |
| Secondary beam | Detector OpticsMount soller |
| path slits | slit 2.5°; Twin_Secondary |
| | motorized slit 5.2 mm |
| Scan mode | Continuous scan |
| Scan type | Locked coupled |
| Step size | 0.02° |
| Time per step | 0.12 second per step |
| Scan range | 3° to 40° |
| Sample rotation speed | 15 rpm |
| Sample holder | Monocrystalline silicon, flat surface |

### Differential Scanning Calorimetry (DSC)

| | |
|---|---|
| Instrument | TA Discovery 2500 and Q2000 |
| Sample pan | Tzero pan and Tzero hermetic lid with |
| | a pin hole of 0.7 mm in diameter |
| Temperature range | 30 to 250° C. or before decomposition |
| Heating rate | 10° C./min |
| Nitrogen flow | 50 mL/min |

### Thermal Gravimetric Analysis (TGA)

| | |
|---|---|
| Instrument | Discovery 5500 and Q5000 |
| Sample pan | Aluminum, open |
| Start temperature | Ambient condition (below 35° C.) |
| Final temperature | 300° C. or abort next segment |
| | if weight <80% (w/w) |
| | (The weight loss of the compound |
| | is no more than 20% (w/w)) |
| Heating rate | 10° C./min |
| Nitrogen flow | Balance 10 mL/min; sample chamber 25 mL/min |

### Dynamic Vapor Sorption (DVS)

| | |
|---|---|
| Instrument | SPSadv-1μ |
| Total gas flow | Max. 4000 ml/min |
| Oven temperature | 25° C. |
| Solvent | Water |
| Method | Cycle: 40-95-0-95-40% RH |
| | Stage Step: 10% |
| | Equilibrium: 240 min for each step |

### Example 1. Slurry Equilibration Screen of Salts of Compound of Formula (I)

About 50 mg of the amorphous compound of Formula (I) and 1 equiv. of acid were added to a screening solvent in a 2-mL glass vial. The mixture was stirred at 50° C. for 2 hours and then at 25° C. for 2 days. The resulting suspension was filtered, dried at 40° C. under vacuum for 2 hours, and solids analyzed by XRPD. The above conditions produced amorphous Formula (I) compound in water, and solutions in methanol using the acids shown in Table 28.

TABLE 28
Acids Evaluated in Slurry Equilibration

| Experiment | Acid |
|---|---|
| 1 | sulfuric acid |
| 2 | 1,2-ethanedisulfonic acid |
| 3 | 1,5-naphthalenedisulfonic acid |
| 4 | phosphoric acid |
| 5 | citric acid |
| 6 | gentisic acid |
| 7 | fumaric acid |
| 8 | Hydrochloric acid |
| 9 | Benzenesulfonic acid |
| 10 | L-malic acid |
| 11 | maleic acid |
| 12 | methanesulfonic acid |
| 13 | succinic acid |
| 14 | ethanesulfonic acid |

### Example 2. Preparation of Hemi 1, 5-Naphthalenesulfonic Acid Salt of Compound of Formula (I)

The clear solution from Experiment 3 of Example 1 was cooled to −20° C. for 3 days to obtain solids. The solids were filtered through a 0.45 μm nylon membrane filter by centrifugation at 14,000 rpm. After drying at 40° C. under vacuum for 2 hr., the solids were analyzed by XRPD. The resulting hemi-1, 5-naphthalenedisulfonate salt Form A gave XRPD peaks as shown in Table 1, and a pattern shown in FIG. 1.

Formula I hemi-1, 5-naphthalenedisulfonate salt Form A gave a differential scanning calorimetry (DSC) pattern as shown in FIG. 2.

Formula I hemi-1, 5-naphthalenedisulfonate salt Form A gave a thermogravimetric analysis (TGA) thermogram as shown in FIG. 3.

Formula I hemi-1, 5-naphthalenedisulfonate salt Form A gave a dynamic vapor sorption (DVS) isotherm as shown in FIG. 4.

### Example 3. Preparation of L-Tartaric Acid Salt of Compound of Formula (I)

Slurry equilibration with Formula I compound and L-tartaric acid according to the method of Example 1 gave Formula I L-tartrate Form A. The Form A crystals gave XRPD peaks as shown in Table 3 and an XRPD pattern according to FIG. 5.

Formula I compound (1 g) and L-tartaric acid (369 mg) were combined in methanol (6 mL) at 50° C. with Formula I L-tartrate seed crystals (5 mg). After stirring at 50° C. for 2 hr, the suspension was allowed to cool to 25° C. and stirred at 25° C. for 5 days. The resulting solids were collected by centrifugation filtration, and then dried at 40° C. under vacuum for about 2 hr. Formula I L-tartrate (1.3 g) Form A was obtained as a light yellow solid (95% yield).

Formula I L-tartrate Form A gave a differential scanning calorimetry (DSC) pattern as shown in FIG. 6.

Formula I L-tartrate Form A gave a thermogravimetric analysis (TGA) thermogram as shown in FIG. 7.

Phase equilibration studies were designed to provide information on a predominant crystal form for phase identification. Formula I L-tartrate Form A was equilibrated in acetonitrile at 50±1° C. To acetonitrile was added Formula I L-tartrate Form A until a cloudy solution was obtained, and then about 30 mg of Formula I L-tartrate Form A was added to the cloudy solution. The mixture was stirred at 50° C.±1° C. for 2 days. The solid was filtered and analyzed by XRPD to give Formula I L-tartrate Form B. The Form B crystals gave an XRPD pattern according to FIG. 8 in an overlay comparison with Form A.

Formula I L-tartrate Form B gave a differential scanning calorimetry (DSC) pattern as shown in FIG. 9.

Formula I L-tartrate Form B gave a thermogravimetric analysis (TGA) thermogram as shown in FIG. 10.

Evaporation studies were carried out to identify the predominant crystal form during uncontrolled precipitation. Saturated solutions of Formula I L-tartrate Form A were prepared by adding Form A to the solvents listed in respectively to give a cloudy solution, and filtering the cloudy solution after stirring for 1-2 hours, after which the solvent was evaporated at 50±1° C. without stirring until a solid was obtained. XRPD was used to study the solid-state morphology of the crystalline forms of the evaporation samples. Table 29 shows a summary of the L-tartrate crystalline forms obtained by these studies.

TABLE 29
Evaporation Studies

| Solvent | Form | XRPD Pattern | DSC | TGA |
|---|---|---|---|---|
| acetone | C | FIG. 11 | FIG. 12 | FIG. 13 |
| 1-propanol | D | FIG. 14 | FIG. 15 | FIG. 16 |
| isopropanol | E | FIG. 17 | FIG. 18 | FIG. 19 |
| Methyl ethyl | F | FIG. 20 | FIG. 21 | FIG. 22 |
| ketone | | | | |

### Example 4. Preparation of Hemi 1, 5-Naphthalenesulfonic Acid Salt of Compound of Formula (I)—Dichloromethane Solvate

About 5 mg of Formula I 1, 5-naphthalenedisulfonate salt Form A from Example 2 was dissolved in 10 mL of dichloromethane. The obtained solutions was filtered through a 0.45 μm syringe membrane filter, and then transferred to a 20-mL glass vial. The vial was covered with a plastic lid with pin holes, and placed in a fume hood at room temperature (about 22-24° C., 70%-74% RH) to allow slow evaporation of the solvent. This vial provided a solid after 34 days, which afforded suitable single crystals for X-ray analysis.

The single crystal structure of the resulting Formula I 1, 5-naphthalenedisulfonate salt dichloromethane (DCM) solvate was determined at 298(2)K. This crystalline form was crystallized in triclinic system, P1 space group with Rᵢₙₜ=4.5% and the final R₁=[I>2σ(I)]=6.6% at 298(2)K. Four molecules of dichloromethane were contained in the asymmetric unit. This DCM solvate was the same polymorphic form as the starting Form A. Single crystal data is summarized in Table 2. An ORTEP image of the DCM polymorph is shown in FIG. 47.

### Example 5. Preparation of Other Salts

Amorphous Formula I (21.1 mg) was slurried in 15 volumes of ethanol:isopropanol (74:26). HCl (1.25 M in isopropanol, 2 equiv., 82.5 μL) was added to the slurry to give complete dissolution. The solutions were stirred overnight to give a white slurry. The solids were filtered and analyzed by XRPD. The wet cake Formula I HCl salt showed XRPD peaks as shown in Table 4, and an XRPD pattern according to FIG. 23. Upon drying, the XRPD pattern had converted to an amorphous form.

Additional salt formations were performed with about 25 mg of amorphous Formula I compound and stock solutions of sulfuric acid prepared in isopropanol. The amorphous Formula I compound was slurried in ethanol or ethyl acetate, and 1.1 equiv. sulfuric acid was added and the mixtures stirred overnight. An additional 1.1 equiv. sulfuric acid was added to the mixture the next day, and stirring continued for an additional day. The slurries were sampled for XRPD analysis.

The wet cake Formula I sulfate salt from ethyl acetate showed XRPD peaks as shown in Table 5, and an XRPD pattern according to FIG. 24. Upon drying, the XRPD was unchanged.

The wet cake Formula I sulfate salt from ethanol showed XRPD peaks as shown in Table 6, and an XRPD pattern according to FIG. 25. Upon drying, the XRPD was unchanged.

While the Formula I sulfate salts were stable on drying, they appeared to deliquesce and turned into a gum after high humidity exposure (>90% relative humidity).

### Example 6. Formula (I) Solvate Preparation

### Slurry

Solvates were produced by slurrying amorphous Formula I compound in a given solvent at room temperature overnight. Solids were collected by filtration and XRPD of the wet cake and dry solids were analyzed. The solids were dried under vacuum at 50° C. for at least 3 hours.

| | Volumes of | XRPD Pattern | |
|---|---|---|---|
| Solvent | solvent | wet | dry |
| MTBE | 45 | FIG. 26 | FIG. 26 |
| Heptane | 54 | FIG. 27 | FIG. 27 |
| Cyclohexane | 53 | FIG. 28 | FIG. 28 |
| methylcyclohexane | 42 | FIG. 29 | FIG. 29 |
| Ethyl acetate | 50 | FIG. 30 | FIG. 30 |
| ethanol | 29 | FIG. 31 | FIG. 31 |
| Isopropanol | 29 | FIG. 32 | FIG. 32 |
| Toluene | 29 | FIG. 34 | FIG. 34 |
| Methyl acetate | 37 | FIG. 35 | FIG. 35 |
| DMF | 14 | FIG. 45 | FIG. 45 |
| THF | 15 | FIG. 38 | FIG. 38 |
| DCM | 14 | FIG. 39 | FIG. 44 |
| acetonitrile | 14 | FIG. 40 | mostly |
| | | | amorphous |
| acetone | 14 | FIG. 41 | FIG. 41 |
| 2-methyltetrahydrofuran | 15 | FIG. 42 | FIG. 42 |
| Methyl ethyl ketone | 14 | FIG. 43 | FIG. 43 |
| NMP | 14 | FIG. 46 | FIG. 46 |

### Anti-Solvent Crystallization

Formula I compound (22 mg) was dissolved at room temperature in 2 volumes DMSO, and subjected to direct anti-solvent addition of water (6 volumes) to give a very thick slurry. The solids were collected by filtration to provide the XRPD peaks according to Table 14 and FIG. 33.

### Solvent Drop Milling

Solvent drop milling was done using a small Wig-L-Bug ball mill with 0.25″ stainless steel ball as milling media. Formula I compound (30 mg) was weighed into a milling capsule and one volume of methyl isobutyl ketone was added. The milling was carried out in 3×30 second increments at 3500 rpm, scraping solids off the capsule walls to minimize caking between millings. The milled samples were analyzed by XRPD as a wet cake. Milling with MIBK gave a new XRPD pattern with peaks as shown in Table 17 and illustrated in FIG. 36. This sample was dried for 3 hr. in a vacuum oven at 50° C. XRPD pattern of the dried sample had peaks as shown in Table 18 and illustrated in FIG. 37.

### Example 7. Formula (I) 1,5-Naphthalenedisulfonate Dichloromethane Solvate Preparation

About 5 mg of Formula (I) 1, 5-naphthalenedisulfonate salt Form A (Table 1, FIG. 1) was dissolved in 10 mL of dichloromethane and sonicated for about 30 sec. Obtained solutions were filtered through a 0.45 μm syringe membrane filter. Obtained clear solutions were transferred to 20 mL glass vials. Then the vials were covered with a plastic lid with pin holds. The vials were placed in a fume hood at room temperature (about 22-24° C., 70%-74% RH) to allow slow evaporation of the solvents. Crystals were obtained after 34 days.

The single crystal structure was determined at 298(2)K. This crystalline form is crystallized in triclinic system, P1 space group with Rᵢₙₜ=4.5% and the final R₁=[I>2σ(I)]=6.6% at 298(2)K. Four molecules of dichloromethane were contained in the asymmetric unit, making this crystal a dichloromethane solvate. The single crystal data is summarized in Table 2. This solvate is the same polymorphic form as the starting Formula (I) 1, 5-naphthalenedisulfonate salt Form A. ORTEP image of the DCM polymorph is shown in FIG. 47.

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A crystalline form of a pharmaceutically acceptable salt or solvate of a compound of Formula (I):

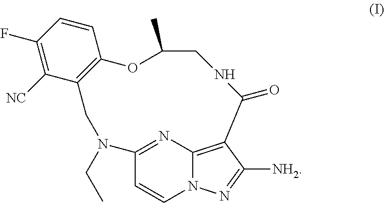 [SMILES: CCN1Cc2c(ccc(F)c2C#N)O[C@@H](C)CNC(=O)c2c(N)nn3ccc1nc23]

2. The crystalline form of claim 1, which is a 1, 5-naphthalenedisulfonic acid salt of a compound of Formula (I).

3. The crystalline form of claim 2, wherein the 1, 5-naphthalenedisulfonic acid salt of a compound of Formula (I) is a hemi 1, 5-naphthalenedisulfonic acid salt of the compound of Formula (I).

4. The crystalline form of claim 1, which is a hydrate.

5. The crystalline form of claim 1, having an X-ray powder diffraction (XRPD) pattern comprising peaks at 6.8, 9.3, and 10.2 degrees 2θ (±0.2 degrees 2θ), wherein the XRPD is made using CuK_{α1} radiation.

6. The crystalline form of claim 1, having an X-ray powder diffraction (XRPD) substantially as set forth in FIG. 1.

7. The crystalline form of claim 1, having a differential scanning calorimetry (DSC) endotherm at about 101° C. and/or about 227° C.

8. The crystalline form of claim 1, having a DSC graph substantially as set forth in FIG. 2.

9. The crystalline form of claim 1, having a thermogravimetric analysis (TGA) thermogram substantially as set forth in FIG. 3 and/or a dynamic vapor sorption (DVS) isotherm substantially as set forth in FIG. 4.

10. The crystalline form of claim 1, which is an L-tartaric acid salt of a compound of Formula (I).

11. The crystalline form of claim 10, having an X-ray powder diffraction (XRPD) pattern comprising peaks at 7.1, 8.2, and 10.5 degrees 2θ (±0.2 degrees 2θ), wherein the XRPD is made using CuK_{α1} radiation.

12. The crystalline form of claim 10, having an X-ray powder diffraction (XRPD) substantially as set forth in FIG. 5.

13. The crystalline form of claim 10, having a differential scanning calorimetry (DSC) endotherms at about 148° C.

14. The crystalline form of claim 10, having a differential scanning calorimetry (DSC) graph substantially as set forth in FIG. 6 and/or a thermogravimetric analysis (TGA) thermogram substantially as set forth in FIG. 7.

15. The crystalline form of claim 1, which is a solvate.

16. The crystalline form of claim 15, wherein the solvate is a dichloromethane (DCM) solvate of the hemi 1, 5-naphthalenedisulfonic acid salt of the compound of Formula (I).

17. The crystalline form of claim 16, characterized by a unit cell as determined by single crystal X-ray crystallography of the following dimensions: a=8.1400 (3) Å; b=11.5875 (5) Å; c=17.4332 (7) Å; α=82.064 (1°); β=84.129 (1°); and γ=81.812 (1°).

18. A pharmaceutical composition comprising a crystalline form of claim 1 and a pharmaceutically acceptable excipient.

19. A process for preparing a crystalline form of claim 4, the process comprising:
(a) combining a compound of Formula (I), 1, 5-naphthalenedisulfonic acid, and an alcohol to form a mixture; and
(b) cooling the mixture;
thereby preparing the crystalline form.

20. A method of treating a cancer in a subject in need thereof, comprising administering to the subject a therapeutically effective amount of a crystalline form of claim 1.
